(19) United States
(12) Patent Application Publication
(10) Pub. No.: US 2014/0134139 A1
(43) Pub. Date: May 15, 2014

(54) CD34+ CELLS AND METHODS OF USE

(71) Applicant: NORTHWESTERN UNIVERSITY, Evanston, IL (US)

(72) Inventors: Raj Kishore, Kildeer, IL (US); Melissa A. Thal, Wheeling, IL (US)

(73) Assignee: NORTHWESTERN UNIVERSITY, Evanston, IL (US)

(21) Appl. No.: 14/074,524

(22) Filed: Nov. 7, 2013

Related U.S. Application Data
(60) Provisional application No. 61/723,505, filed on Nov. 7, 2012.

(51) Int. Cl. A61K 35/44

(52) U.S. Cl. CPC A61K 35/44
USPC 424/93.7; 435/377

(57) ABSTRACT

Disclosed herein is a reprogrammed endothelial progenitor cell, said cell comprises a bone marrow-derived cell expressing the CD34+ marker and at least one cardiomyocyte-specific gene, as well as methods for preparing and using the same for cardiac regenerative medicine.

### CROSS-REFERENCE TO RELATED APPLICATIONS

This application claims benefit of priority under 35 U.S.C. 119 to U.S. provisional patent application Ser. No. 61/723,505 filed Nov. 7, 2012, and entitled “CD34+ CELLS AND METHODS OF USE,” the contents of which are herein incorporated by reference in its entirety.

### STATEMENT REGARDING FEDERALLY SPONSORED RESEARCH OR DEVELOPMENT

This invention was made with government support under HL107093, HL091983, HL105597, HL095874, HL053354 and HL108795 awarded by the National Institutes of Health. The government has certain rights in the invention.

### BACKGROUND

1. Technical Field

The present disclosure relates to progenitor cell based therapies for cardiac regenerative medicine. In particular, the CD34+ cells and modification thereof for their conversion into cardiomyocytes in methods for cardiac disease therapy are disclosed.

2. Description of Related Art

Despite the pervasive belief that the heart has limited regenerative capacity, progenitor-cell based therapy has been shown to provide substantial clinical benefits for ischemic diseases such as chronic angina, acute myocardial infarction and heart failure (Britten M B et al., Circulation 108:2212-2218 (2003); Losordo D W et al., Circulation 109:2487-2491 (2004); Losordo D W et al., Circulation 109:2692-2697 (2004); Schachinger V et al., Eur. Heart J. 27:2775-2783 (2006); Strauer B E et al., Circulation 106:1913-1918 (2002); Losordo D W et al., Circ. Res. 109:428-436 (2011)). Specifically, murine endothelial progenitor cells (“EPC”; Krasinski K et al., Circulation 95:1768-1772 (1997)), and the human equivalent CD34+ mononuclear cells are capable of homing to infarct and peri-infarct myocardium upon ischemia, inducing angio/vasculogenesis, and augmenting cardiac function and survival though paracrine mediated growth factor secretion (Kinnaird T et al., Circulation 109:1543-1549 (2004); Kawamoto A et al., Trends Cardiovasc. Med. 18:33-37 (2008); Pesce M et al., Pharmacol. Ther. 129:50-61 (2011)). Although the re-vascularization appears to result in real improvements in quality of life, the ultimate goal of cardiovascular regenerative medicine is to regenerate lost myocytes in addition to neovasculature. There is no convincing evidence that EPCs have cardiomyocyte differentiation potential (Balsam L B et al., Nature 428:668-673 (2004); Murry C E et al., Nature 428:664-668 (2004)). Also, since the EPCs from aged patients with existing metabolic diseases and cardiovascular risk factors are known to have diminished functional properties, epigenetic modification of EPC may lead to an enhancement in their phenotypic and functional properties thereby further augmenting the clinical relevance of this cellular therapy (Vasa M et al., Circ. Res. 89:E1-7 (2001); Loomans C J et al., Diabetes 53:195-199 (2004); Michowitz Y et al., Heart 93:1046-1050 (2007)). Previous attempts to improve upon EPC therapy involve pretreatment with small molecules or gene therapy, requiring introduction of exogenous DNA (Seeger F H et al., Nat. Clin. Pract. Cardiovasc. Med. 4 Suppl 1:S110-113 (2007)). In all previous studies, improvements have been limited to incremental enhancements of previously characterized therapeutic effects: survival, homing, proliferation, and paracrine factor release because they only target one gene or one signaling pathway. Importantly, none of these improved strategies have conferred enhanced differentiation capacity.

The epigenetic code, including post-translational modifications in histones, is responsible for adding complexity to gene regulation beyond sequence specificity. It plays a critical role in regulating everything from gene expression, cell cycle, cell fate, differentiation and ultimately the capacity for trans-differentiation in vivo (Trojer P et al., Cell 125:213-217 (2006); Jenuwein T et al., Science 293:1074-1080 (2001)). Endothelial cells, vascular smooth muscle cells and cardiomyocytes all differentiate from a common progenitor in the mesoderm, suggesting that reprogramming endothelial cells to an earlier state in mesodermal development could recapitulate their cardiomyogenic potential. The extant art in the field does not provide sufficient details to enable a skilled artisan to determine which cells are amenable for cell reprogramming and the means for achieving such cell reprogramming.

### SUMMARY

In a first respect, a reprogrammed endothelial progenitor cell, said cell includes a bone marrow-derived cell expressing the CD34+ marker and at least one cardiomyocyte-specific gene is disclosed. In one aspect, the reprogrammed endothelial progenitor cell lacks teratoma potential.

In a second respect, a method of reprogramming an endothelial progenitor cell is disclosed. In this respect, method includes the step of contacting a bone marrow-derived, CD34+ cell with a first reprogramming agent. In one aspect of this method, the first reprogramming agent includes an epigenetic modifier. In a further aspect of this method, the epigenetic modifier is selected from the group consisting of Trichostatin A, valproic acid, 5′-Azacytidine and BIX-01294. In a second aspect, the method further includes a second reprogramming agent. In a third aspect, the method further includes the step of forming an admixture of the first and second reprogramming agents prior to contacting the bone marrow-derived, CD34+ cell. In an alternative third aspect, the method specifies that the bone marrow-derived, CD34+ cell is contacted with the first programming agent for a first time period, followed by the bone marrow-derived, CD34+ cell being contacted with the second reprogramming agent for a second time period. In a further aspect of the method, the first reprogramming agent comprises valproic acid and the second reprogramming agent comprises 5′-Azacytidine. In certain embodiments of the method, the bone marrow-derived, CD34+ cell is contacted with from about 1.0 mM to about 5.0 mM valproic acid ranging from about 1 hr to about 48 hr followed by the addition of from about 250 nM to about 1.0 mM 5′-Azacytidine ranging from about 1 hr to about 48 hr. In yet other embodiments of the method, the bone marrow-derived, CD34+ cell is contacted with about 2.5 mM valproic acid for about 24 hr followed by the addition of about 500 nM to 5′-Azacytidine ranging for about 24 hr. In a further aspect of this method, additional steps of isolating the bone marrow-derived, CD34+ cell from bone marrow-derived population of cells with a cell sorting technique are disclosed. In one embodiment of an aspect of this method, the cell sorting technique is fluorescent activated cell sorting.

In a third respect, method of improving an ischemic condition is disclosed. The method includes the step of contacting the ischemic condition with a therapeutically effective amount of reprogrammed endothelial progenitor cells. These cells include bone marrow-derived cells expressing the CD34+ marker and at least one cardiomyocyte-specific gene. In one aspect, the method specifies that the ischemic condition is selected from an ischemic-damaged non-cardiotissue or an ischemic-damaged myocardium. In one embodiment, the method specifies the ischemic condition comprises an ischemic-damaged myocardium. In a further aspect of this method, the therapeutically effective amount of reprogrammed endothelial progenitor cells comprises an amount from about 1×10⁴ cells to about 1×10⁶ cells per kg body weight. In a further aspect of the method, the therapeutically effective amount of reprogrammed endothelial progenitor cells are administered by injection.

In a fourth respect, a pharmaceutical composition is disclosed. The pharmaceutical composition includes a population of reprogrammed endothelial progenitor cells comprising bone marrow-derived cells expressing the CD34+ marker and at least one cardiomyocyte-specific gene and a physiologically acceptable buffer.

In a fifth respect, a kit for preparing a reprogrammed endothelial progenitor cell, wherein said cell comprising a bone marrow-derived cell expressing the CD34+ marker and at least one cardiomyocyte-specific gene is disclosed. The kit includes reprogramming agents and optionally instructions.

In a sixth respect, a method of reprogramming a diseased cell to its normal functioning counterpart is disclosed. The method includes contacting the diseased cell with a first reprogramming agent. In a first aspect of this method, the diseased cell includes a diabetic cell. In a second aspect, this method specifies that the normal functioning counterpart includes the activation of an angiogenic gene. In a third aspect, the method further includes a second reprogramming agent. In a fourth aspect, the method specifies that the first and second reprogramming agents include epigenetic modifiers. In a fifth aspect of the method, the epigenetic modifiers are selected from the group consisting of Trichostatin A, valproic acid, 5′-Azacytidine and BIX-01294. In a sixth aspect, the method further includes the step of forming an admixture of the first and second reprogramming agents prior to contacting the diseased cell. In a seventh aspect of the method, the diseased cell is contacted with the first programming agent for a first time period, followed by the diseased cell being contacted with the second reprogramming agent for a second time period. In an eighth aspect of the method, the first reprogramming agent includes valproic acid and the second reprogramming agent comprises 5′-Azacytidine. In an ninth aspect of the method, the diseased cell is contacted with from about 1.0 mM to about 5.0 mM valproic acid ranging from about 1 hr to about 48 hr followed by the addition of from about 250 nM to about 1.0 mM 5′-Azacytidine ranging from about 1 hr to about 48 hr. In a tenth aspect of the method, the diseased cell is contacted with about 2.5 mM valproic acid for about 24 hr followed by the addition of about 500 nM to 5′-Azacytidine ranging for about 24 hr.

In a seventh respect, a method to restoring an aged cell having reduced functional competency to increased functional competency for treating an ischemic condition is disclosed. The said method includes the step of contacting the aged cell with a first reprogramming agent. In a first aspect of the method, the ischemic condition is selected from the group consisting of peripheral artery disease, ischemic myocardial tissue, ischemic brain tissues and ischemic wounds. In a second aspect the method further includes a second reprogramming agent. In a third aspect of the method, the first and second reprogramming agents comprises epigenetic modifiers. In a fourth aspect of the method, the epigenetic modifiers are selected from the group consisting of Trichostatin A, valproic acid, 5′-Azacytidine and BIX-01294. In a fifth aspect, the method further includes the step firming an admixture of the first and second reprogramming agents prior to contacting the aged cell. In a sixth aspect, the method specifies that the aged cell is contacted with the first programming agent for a first time period, followed by the aged cell being contacted with the second reprogramming agent for a second time period. In a seventh aspect of the method, the first reprogramming agent includes valproic acid and the second reprogramming agent comprises 5′-Azacytidine. In an eighth aspect of the method, the aged cell is contacted with from about 1.0 mM to about 5.0 mM valproic acid ranging from about 1 hr to about 48 hr followed by the addition of from about 250 nM to about 1.0 mM 5′-Azacytidine ranging from about 1 hr to about 48 hr. In an ninth aspect of the method, the aged cell is contacted with about 2.5 mM valproic acid for about 24 hr followed by the addition of about 500 nM to 5′-Azacytidine ranging for about 24 hr.

These and other features, objects and advantages of the claimed subject matter provided in this disclosure will become better understood film the description that follows. In the description, reference is made to the accompanying drawings, which form a part hereof and in which there is shown by way of illustration, not limitation, embodiments of the claimed subject matter.

### BRIEF DESCRIPTION OF THE DRAWINGS

FIG. 1A depicts isolation of Lineage-Sca-1+CD31+ EPCs from mouse bone marrow wherein a sorting strategy for obtaining EPCs directly from mouse bone marrow. Subpanel (i): first cell sorting for isolation of lineage negative cells by depletion of cells positive for committed lineages from input bone marrow cells; subpanel (ii): second cell sorting for Lineage+ cells that are also positive for Stem cell Antigen 1 (Sca1); and subpanel (iii): final cell sorting of defined target cells which are lineage (−)/Sca1(+)/CD31(+). This final sorted cell population is designated as EPC. Numerical values shown in subpanels (i)-(iii) are the percentages of total bone marrow in the delineated areas.

FIG. 1B depicts angiogenic activity of EPC in tube formation assay. DiI labeled Lin-Sca-1+CD31+ cells incorporate into tubes formed by 5.0×10⁴ SVEC cells (10× magnification), wherein subpanel (i): identifies the DiI labeled EPCs by fluorescence microscopy (bottom panel); subpanel (ii): depicts better tubular architecture of SVECs where EPCs were added (bottom panel) compared to SVECs without EPCs (top panel); and subpanel (iii): shows a merged picture indicating that labeled EPCs incorporate into tubes formed by SVECs.

FIG. 1C depicts an exemplary drug treatment of sorted EPCs induces gene expression of pluripotency genes (Oct4, Nanog and Sox2) compared to the untreated cells based on real-time PCR analysis. Values are all fold change compared to the untreated cells. (n=3).

FIG. 1D depicts an exemplary drug treatment of sorted EPCs induces gene expression of cardiomyocyte genes (Nkx2.5, Cx43 and Tnnt2) compared to the untreated cells based on real-time PCR analysis. Values are all fold change compared to the untreated cells. (n=3).

FIG. 2A depicts real-time PCR data represented as fold difference in mRNA expression in 5.0×10⁵ VPA then 5′ Aza treated CD34+ cells compared to untreated control cells (n=3).

FIG. 2B depicts a clustering analysis of microarray data comparing human CD34+ cells to those treated with VPA/5′ Aza. Up- and down-regulated genes are represented in red and green colors, respectively. CD34+ cells from two healthy patient donors were run in triplicate for both conditions.

FIG. 2C depicts a pie graph depicting pattern of expression changes of statistically significantly affected genes upon VPA/5′ Aza treatment of CD34+ cells.

FIG. 2D depicts an exemplary drug treatment of sorted EPCs induces gene expression of endothelial genes (eNOS and VE cadherin) compared to the untreated cells based on real-time PCR analysis. Values are all fold change compared to the untreated cells. (n=3).

FIG. 3 depicts results that reveal no teratoma formation occurred in nude mice when the animals injected in the flanks with 1 million untreated bone marrow cells (subpanel (i)), VPA/5′ Aza-treated bone marrow cells (subpanel (ii)) or BIX-01294-treated bone marrow cells (subpanel (iii)). Subpanel (iv) illustrates positive teratoma formation with 1 million mouse embryonic stem cells (mES) (n=3).

FIG. 4A shows exemplary effects of VPA/5′ Aza or BIX-01294 treatment on histone modifications in ECs, wherein Western blot analysis of acetylated H3K9, or pan acetylated H4 are illustrated.

FIG. 4B depicts an exemplary quantitative assessment of modified histone levels relative to total histone levels of data in FIG. 4A. Values are fold change compared to untreated SVEC cells. Statistical data key: NS, not significant; *, p≦0.05 (n=3).

FIG. 4C shows exemplary effects of VPA/5′ Aza or BIX-01294 treatment on histone modifications in ECs, wherein di-methyl H3K9 levels from 10 million SVEC cells after 24 hours treatment with 1 mM VPA followed by an additional 24 hours with 500 nM 5′ Azacytidine or 1 μM BIX-01294 are illustrated.

FIG. 4D depicts an exemplary quantitative assessment of modified histone levels relative to total histone levels of data in FIG. 4C. Values are fold change compared to untreated SVEC cells. Statistical data key: NS, not significant, **, p≦0.01 (n=3).

FIG. 5A depicts an epigenetically reprogrammed EPCs having increased aCH3K9 associated with cardiac-specific promoters, wherein an exemplary ChIP assay at CMC-associated gene promoters and 2 regulatory regions of Oct4 CP=core promoter, RP=regulatory promoter is illustrated. Values represent percent of total input. Statistical data key: *, p<0.01.

FIG. 5B depicts an epigenetically reprogrammed EPCs having increased aCH3K9 associated with cardiac-specific promoters, wherein an exemplary pyro-sequencing of the Nkx2.5 promoter from isolated and bisulfite converted genomic DNA from SVECs with either no drug, VPA/5′ Aza or BIX-01294 is illustrated. Converted NIH-3T3 gDNA was used as a positive control.

FIG. 6A illustrates that drug-contacted mouse EPCs improve left ventricular function post myocardial infarction (AMI) following injection of EPCs, wherein echocardiognaphic analysis of left ventricular heart function assessed by percent fractional shortening (subpanel (i)) and percent ejection fraction (subpanel (ii)) prior to surgery and at 7, 14 and 28 days post-surgery is shown. Data key for injected materials: MI, saline negative control; EPC=Lin-Sca-1+CD31+ mouse bone marrow cells; EPC+VPA/5 Aza, EPCs contacted with VPA/5′ Aza; EPC+BIX-01294, EPCs contacted with BIX-01294. Statistical data n≧6 at each time point; p<0.05 EPC vs. EPC+5Aza/VPA and EPC vs. EPC+BIX01294.

FIG. 6B illustrates that drug-contacted mouse EPCs improve left ventricular function post myocardial infarction (AMI), wherein left ventricle end-diastolic diameter (LVEDD, subpanel (i)) and left ventricle end-systolic diameter (LVESD, subpanel (ii)) measured from short-axis m-mode echocardiography is illustrated. Data key for injected materials as in FIG. 6A. Statistical data key: *, p≦0.05, **, p≦0.01 (n≧6).

FIG. 7A depicts a representative histological evaluation of infracted hearts that indicates drug treated EPCs confer less severe disease and allow for CMC trans-differentiation in vivo, wherein Masson's trichrome-stained heart sections (28 d post-AMI) are illustrated. Scale bar is 5 mm. Subpanel key is MI, animal hearts injected with saline as a negative control; EPCs, animal hearts injected with untreated EPCs (Lin-Sca-1+CD31+ mouse bone marrow cells); EPCs+BIX-01294, animal hearts injected with EPCs contacted with BIX-011294; and EPCs+VPA/5′ Aza, animal hearts injected with EPCs contacted with 5′ Aza and VPA.

FIG. 7B depicts an exemplary quantitative analysis of infarct size of infracted hearts of FIG. 7A. Quantification is depicted as % parameter changes in treatment groups when compared to control group (MI, animal hearts injected with saline as a negative control). Subpanel key as in FIG. 7A. Statistical data key: **, p≦0.01 (n≧3).

FIG. 7C depicts an exemplary quantitative analysis of fibrotic area (% LV area) at 28 d post-AMI. Quantification is depicted as % parameter changes in treatment groups when compared to control group (MI, animal hearts injected with saline as a negative control). Subpanel key as in FIG. 7A. Statistical data key: *, p≦0.05, (n≧3).

FIG. 7D depicts number of capillaries in three exemplary high power fields per heart of the border zone of myocardial infarcted mice. Subpanel Key is EPCs, animal hearts injected with untreated EPCs (Lin-Sca-1+CD31+ mouse bone marrow cells); EPCs+BIX-01294, animal heats injected with EPCs contacted with BIX-01294; and EPCs+VPA/5′ Aza, animal hearts injected with cells contacted with 5-Aza and VPA.

FIG. 7E depicts results of capillary density per mm² as calculated from representative fields as illustrated in FIG. 7C for minimally three mice per condition. Statistical data key; **p<0.01.

FIG. 7F depicts IF of heart sections at day 14 post-AMI for GFP and alpha-sarcomeric actin. DAPI was used to stain nuclei. Subpanel key as in FIG. 7A. Arrows point to double stained cells identified as cardiomyocytes. Representative images shown of n=4 animals.

FIG. 8A depicts drug-contacted human CD34+ cells (when used as sub-therapeutic dose of 25,000) improve LV function of immune-deficient mice after ML based on %/FS (subpanel (i)) and % EF (subpanel (ii)), wherein echocardiographic analysis at baseline and at days 7, 14 and 28 post-AMI for all groups. Data key for injected materials: saline, animal hearts injected with saline as a negative control; 25K CD34, animal hearts injected with human CD34+ EPCs (2.5×10⁴); 25K CD34 VPA/5′ Aza, animal hearts injected with human CD34+ EPCs (2.5×10⁴) contacted with VPA/5′ Aza.

FIG. 8B depicts Masson's trichrome stained 5 μm sections 1 mm below suture of infracted hearts 28 days post-AMI to assess fibrosis (stained blue) and LV wall-thickness. Animal hearts injected with saline as a negative control (subpanel (i)); animal hearts injected with human CD34+ EPCs (2.5×10⁴) (subpanel (ii)); and animal hearts injected with human CD34+ EPCs (2.5×10⁴) contacted with VPA/5′ Aza (subpanel (iii)). Scale bar is 5 mm.

FIG. 8C depicts quantifications of measurements from Masson's trichrome stained sections of FIG. 8B for infarct size as a percent of the circumference. Key: MI, animal hearts injected with saline as a negative control; CD34, animal hearts injected with human CD34+ EPCs (2.5×10⁴); and CD34+ VPA/5′ Aza, animal hearts injected with human CD34+ EPCs (2.5×10⁴) contacted with VPA/5′ Aza.

FIG. 8D depicts exemplary capillary density measurements as calculated per mm² from 3 high-powered fields, wherein (n≧5) for each condition was assessed. Subpanel key as in FIG. 8C. Statistical data key: *, p≦0.05.

FIG. 9A depicts VPA/5′ Aza contacted CD34+ cell therapy in mouse AMI results in less apoptosis and increased proliferation in the border zone, wherein representative TUNEL stained sections with alpha-sarcomeric actin (red) and DAPI (blue) for CD34+ EPCs contacted without any epigenetic modifiers (subpanel (i)) or CD34+ EPCs contacted with VPA/5′ Aza (subpanel (ii)). Scale bar is 20 μm. Quantification of TUNEL+ cells from 3 high power fields per heart of the border zone of myocardial infarcted mice, minimally 4 mice per condition, 14 days post-AMI (subpanel (iii)). For subpanel (iii), statistical data key: *, p≦0.05.

FIG. 9B depicts quantification of Ki67+ nuclei per high power field (HPF) at day 28 post-AMI. Data key for injected materials: saline, animal hearts injected with saline as a negative control; 25K CD34, animal hearts injected with human CD34+ EPCs (2.5×10⁴); 25K CD34 VPA/5′ Aza, animal hearts injected with human CD34+ EPCs (2.5×10⁴) contacted with VPA/5′ Aza. Statistical data key: *, p≦0.05.

FIG. 10 depicts reprogrammed human CD34+ cells have increased angiogenic protein secretion. Human angiogenesis ELISA array shows a trend of increased angiogenesis protein levels in conditioned media from VPA/5′ Aza-contacted CD34+ cells compared to the untreated cells. Representative results from one array, confirmed by an independent experiment.

FIG. 11 depicts in vivo cardiomyocyte differentiation identified by immunofluorescence in d7 hearts of mice receiving AMI and DiI labeled CD34+ (subpanel (i)) or VPA/5′ Aza treated CD34+ cells (subpanel (ii)) in the border zone. DiI labeled donor cells (red), alpha-sarcomeric actin 48 and nuclei (DAPI). White arrow indicates potential donor-derived cardiomyocyte and shown bigger in inset. Scale bar represents 20 mm. Representative images from each group are shown.

FIG. 12 depicts drug treatment rescues angiogenic dysfunction in diabetic EPCs. Subpanel (i): tube formation activity of HUVEC cells (positive control); subpanel (ii): tube formation of EPCs obtained from the bone marrow of healthy wild-type mice; subpanel (iii): drug treatments (5′-Aza?VPA enhances tube formation by wild type healthy EPCs; subpanel (iv): EPC from diabetic mice are functionally deficient in tube formation, subpanel (v): drug-treatment of diabetic EPCs rescues their ability to form tubes; and subpanel (vi): quantification of tube formation activity depicted in panels i-v. Statistical data key: ***, p<0.05; *, p<0.001.

FIG. 13 depicts results of experiments showing expression of Pro-angiogenic RNA increased in Diabetic EPCs after drug treatments. Subpanel (i): increased expression of angiopoetin 1 subpanel (ii): increased expression of angiopoetin 2: subpanel (iii): increased expression of endothelial Nitic Ooxide Synthase (eNOS); subpanel (iv): increased expression of insulin growth factor 1; subpanel (v): increased expression of Vascular endothelial cadherin (VE Cad); and subpanel (vi): increased expression of vascular endothelial growth factor (VEGF). Statistical data key: *, p<0.05.

FIG. 14 depicts results of experiments showing treated diabetic EPCs restore blood flow to ischemic hind limbs of diabetic mice. Subpanel (i): loss of ischemic limb (circled) after hindlimb ischemia in diabetic mouse as assayed by laser doppler perfusion imaging to quantify blood flow. Brighter colors indicate high blood blow, darker colors indicates less blood flow (see scale); subpanel (ii): diabetic EPCs injected in ischemic hindlimb of diabetic mouse fail to completely restore blood flow and limb loss; subpanel (iii): diabetic EPCs contacted with 5′ Aza/VPA significantly increase blood flow and increased limb salvage; subpanel (iv): color scale used for blood flow assessment in laser Doppler perfusion imaging; and subpanel (v): quantification of ratio of blood flow in ischemic limb vs. control uninjured limb.

FIG. 15 depicts results of experiments showing ischemic limb capillary density increases in mice receiving treated diabetic EPCs. Subpanel (i): quantification of capillary density in ischemic hindlimbs injected with untreated diabetic EPCs vs. drug-treated Diabetic EPCs; subpanel (ii): representative tissue staining for capillaries in mice receiving untreated diabetic EPCs; and subpanel (iii): representative tissue staining for capillaries in mice receiving drug-treated diabetic EPCs.

FIG. 16 depicts results of experiments of intramuscle injection of untreated diabetic EPCs (subpanel (i)) and treated diabetic EPCs (subpanel (ii)) in animals having ischemic limbs that demonstrates that treated diabetic EPCs preserve muscle architecture in the ischemic limbs.

FIG. 17A depicts results of protein array analyses of condition medium obtained from VPA/5′ Aza-contacted CD34+ cells (subpanel (i)) or untreated CD34+ cells (subpanel (ii)).

FIG. 17B depicts relative expression level of angiogenic cytokines in condition medium obtained from VPA/5′ Aza-contacted CD34+ cells (blue bars) or untreated CD34+ cells (red bars).

### DETAILED DESCRIPTION

Cells and methods are disclosed that remove inhibitory epigenetic modifications in both mouse and human EPCs, thereby conferring enhanced therapeutic potential of acute myocardial infarction. Not only is the inherent paracrine activity greater as evidenced by improved capillary density, cell survival, and proliferation within the border zone of the infarct but also the modified cells acquire cardiomyogenic potential. Though not intended to limit the scope of the claimed subject matter, the mechanism for the enhanced functionality and differentiation potential is the positive effect epigenetic modifying drugs have on global gene transcription, which primes the cell to respond to environmental stimuli. The methods can provide a clinically effective way of modifying an existing cellular therapy with potentially significant improvements not only in revascularization of the ischemic tissue but also in regeneration of the damaged myocardium.

Endothelial progenitor cells can be obtained from ex vivo culture of unfractionated peripheral blood mononuclear cells or bone marrow and expansion in endothelial specific media. Endothelial progenitor cells are preferably obtained from bone marrow by suitable techniques known in the art. The CD34+ subpopulation of EPCs are fractionated from bulk EPCs by any one of a number of acceptable sorting techniques, such as fluorescent activated cell sorting, magnetic cell selection and single cell sorting. Preferably, the CD34+ EPC population is isolated from bulk EPCs using fluorescent activated cell sorting (FACS). The CD34+ EPC population can be cultured ex vivo using in vivo cell culturing techniques well known in the art. For example, human CD34+ cells can be cultured in endothelial specific media having defined properties, such as serum-free hematopoietic cell media containing the requisite growth factor and nutrient supplements. A preferred culture media for CD34+ EPCs is X-VIVO (Lonza) media supplemented with 0.5% human serum albumin, 50 ng/mL Flt3 ligand, 20 ng/mL stem cell factor, 50 ng/mL VEGF, and 10 ng/mL thrombopoietin.

To promote cellular reprogramming, the EPCs can be contacted with one or more reprogramming agents. Preferably, the reprogramming agents include epigenetic modifiers. Exemplary reprogramming agents as epigenetic modifiers include Trichostatin A, valproic acid (“VPA”), 5′-Azacytidine (“5′-Aza” or “5′Aza”) and BIX-01294, among others. The EPCs can be contacted with a plurality of reprogramming agents, either taken in combination or taken in sequential succession. For a method of contacting EPCs with a plurality of reprogramming agents taken in combination, the reprogramming agents are preferably formed as an admixture prior to contacting the EPCs. For a method of contacting EPCs with a plurality of reprogramming agents taken in sequential succession, the EPCs are contacted initially with a first reprogramming agent for a first period of time and then the EPCs are contacted with a second reprogramming agent for a second period of time. For a method of contacting the EPCs with a plurality of reprogramming agents taken in sequential succession, such as contacting EPCs with a first reprogramming agent for a first period of time and then a second reprogramming agent for a second period of time, the first and second period of times of contacting the EPCs can either overlap or not overlap. In the case where the first a second period of times of contact overlap, the second reprogramming agent can be added to culture media containing the first reprogramming agent in contact with the EPCs. In the case where the first a second period of times of contact do not overlap, the second culture media containing the first reprogramming agent in contact with the EPCs is removed from the EPCs culture and fresh culture media containing the second reprogramming agent is added to the EPCs culture.

The amount or concentration of a reprogramming agent for contacting an EPC culture is based upon a wt/vol (%) or a concentration of the reprogramming agent in the culture media in contact with the EPC culture. For reprogramming agents of any variety, the optimal amount or concentration can be readily determined by empirical assessment of changes epigenetic program expression, molecular cues of increased molecular plasticity and induction of cardiomyocyte specific gene expression, as further exemplified by the working examples presented in this disclosure. For the reprogramming agents that include epigenetic modifiers like valproic acid (“VPA”) 5′-Azacytidine (“5′-Aza” or “5′ Aza”), and BIX-01294, the optimal concentrations for directing reprogramming of EPCs and their subsequent differentiation to cardiomyocytes are as follows. A preferred concentration of valproic acid as a reprogramming agent is from about 0.5 mM to about 10.0 mM. A preferred concentration of 5′-Azacytidine as a reprogramming agent is from about 200 nM to about 5.0 μM. A preferred method for reprogramming EPCs to cardiomyocytes is to contact an EPC culture with culture media containing from about 1.0 mM to about 5.0 mM valproic acid as a first reprogramming agent for a first period of time ranging from about 1 hr to about 96 hr followed by the addition of from about 250 nM to about 1.0 μM 5′-Azacytidine as a second reprogramming agent to the same culture media for a second period of time ranging from about 1 hr to about 96 hr. A highly preferred method for reprogramming EPCs to cardiomyocytes is to contact an EPC culture with culture media containing about 2.5 mM valproic acid as a first reprogramming agent for a first period of time ranging of about 24 hr followed by the addition of about 500 nM 5′-Azacytidine as a second reprogramming agent to the same culture media for a second period of time of about 24 hr.

A preferred concentration of BIX-01294 as a reprogramming agent is from about 1 μM to about 1 mM in contact with cultured EPCs, wherein the period of contact is from about 1 hr to about 72 hr. It should be noted, however, that BIX-01294 is toxic to human CD34+ EPCs and is not recommended for reprogramming human CD34+ cells in this disclosure.

As explained previously. EPC cultures can be contacted simultaneously with an combination of preferred concentrations of reprogramming agents, such as that formed as an admixture prior to addition to culture medium in contact with the EPCs. Alternatively, EPCs can be contacted at different times, such as in a staggered arrangement, with preferred concentrations of reprogramming agents added to the culture medium in contact with the EPCs.

Without the claimed subjected matter being bound by any particular theory, these epigenetic modifiers remove the transcription-restrictive epigenetic marks and harness all the differentiation potential the endothelial progenitor cell might possess. The removal of epigenetic repressive marks by these compounds can enable remodeling of the chromatin surrounding cardiomyocyte specific genes (as well as global genes), thereby providing a window wherein the cellular epigenome is permissive to desired gene transcription and potentially trans-differentiation when cells are cultured under appropriate culture conditions or exposed to the proper microenvironment in vivo.

Available Cell Types from Subjects Amenable for Reprogramming

The CD34+ EPCs can be obtained from a variety of subjects, wherein the cells are preferably obtained from the bone marrow of the subjects for reprogramming with the reprogramming agents disclosed herein. In particular, bone marrow from healthy subjects, subjects having a disease (for example, subjects having a form a diabetes), or elderly subjects are amenable for reprogramming with the disclosed reprogramming agents. For example, bone marrow from a subject having a disease can be used as a source to obtain a diseased cell having dysfunctional phenotype relative to the normal cell counterpart. The diseased cell can be reprogrammed by contacting the diseased cell with the selected reprogramming agents and methods disclosed herein. Likewise, bone marrow from an elderly subject can be used as a source to obtain an aged cell having reduced functional competency. The aged cell can be restored to a cell of increased functional competency by contacting the aged cell with the reprogramming agents and methods disclosed herein. Once aged cells are restored with increased functional competency, the cells can be used to treat an ischemic condition in a subject. Preferred ischemic conditions can be selected from the group consisting of peripheral artery disease, ischemic myocardial tissue, ischemic brain tissues and ischemic wounds.

### Treatment Modalities

Subjects in need of the reprogrammed EPCs of the present disclosure include those having ischemic tissue and/or damaged myocardium. A therapeutically effective amount of reprogrammed EPCs can be administered directly via direct tissue injection (for example, intra-myocardial injection) at the ischemic border zone. The reprogrammed EPCs can be resuspended in any physiologically acceptable buffer, such as phosphate buffered saline. Preferred doses of EPCs per injection fall within the range from about 1×10⁴ cells to about 1×10⁶ cells/kg body weight, including variations of cell amounts within this range. A therapeutically effective amount of either mouse or human CD34+ cells EPCs reprogrammed according to the methods presented herein provide increased ischemic angiogenesis in vivo, including the enhancement of both capillary density and the number of arterioles in the border zone of the infarct, suggesting both angiogenic and vasculogenic activity and myocardial repair capabilities as evident from improved LV functions and anatomical repair including reduced fibrosis and apoptosis following acute myocardial infarction in mice. Generally, the methods are used for treating subjects with autologous cell sources, such as those having histocompatible and immunologically tolerant phenotypes.

The working examples provided in this disclosure demonstrates that that small molecule mediated epigenetic reprogramming of EPCs, both mouse and human, significantly enhances both their angiogenic and functional activity as well as lead to a cellular epigenome that is conducive to a more plastic phenotype capable of trans-differentiation into cardiomyocyte lineage. Several lines of evidence support this conclusion: 1) treatment of both mouse EPC and human CD34+ cells with small molecule inhibitors of DNA methyltransferase, histone deacetylase and G9a histone methyltransferase leads to globally up-regulated gene transcription indicative of dynamic chromatin remodeling and transcription permissive epigenome; 2) cells treated with these small molecules show de novo induction of both cardiomyocyte specific genes and yet retain their endothelial gene expression; 3) epigenetically reprogrammed EPCs show enhanced angiogenic activity both in terms of paracrine factor secretion and increased ischemic angiogenesis in vivo, including the enhancement of both capillary density and the number of arterioles in the border zone of the infarct, suggesting both angiogenic and vasculogenic activity; 4) epigenetic reprogramming of both mouse and human CD34+ cells significantly enhances their myocardial repair capabilities as evident from improved LV functions and anatomical repair including reduced fibrosis and apoptosis following acute myocardial infarction in mice; and finally 5) reprogrammed cells differentiate into cardiomyocytes, in vivo. Of important note is the fact that the selected epigenetic modifiers improve reprogramming efficiency, while being insufficient to induce pluripotency, thereby eliminating the risk of teratoma formation associated with cell therapy in a novel approach. The disclosure therefore has teachings with clear and direct translational bearing on the bone marrow-derived EPC based cellular therapies for myocardial regenerative medicine.

### Kits

In another aspect, kits are provided for preparing reprogrammed EPCs for their use as described herein. A kit can include reprogramming agents, tissue culture media and materials, immunohistochemical stains and other reagents for detecting epigenetic marker molecular phenotype, and instructions.

### EXAMPLES

The disclosure will be more fully understood upon consideration of the following non-limiting examples, which are offered for purposes of illustration, not limitation.

### Example 1

### Animals, Materials and Methods

### Mice

Eight-ten week old C57BL/6J (stock number 000664). C57BL/6-Tg eGFP (stock number 003291) or nude (stock number 00819) mice were purchased from Jackson Laboratory. All experiments conform to protocols approved by the Institutional Animal Care and Use Committee at Northwestern University (Chicago, Ill.) in compliance with all state and federal regulations governing the use of experimental animals.

### FACS Sorting

Bone marrow extracted from the femurs, tibiae and hip-bones of 10-12 week old C57BL/6J or eGFP transgenic mice were stained with labeled antibodies against Lineage (CD3e(145-2C11), CD11b(M1/70), B220(RA3-6B2), Ter119(Ly76), Ly6G/C(RB6-8C5)) Sca-1 (D7) and CD31 (MEC13.3) then sorted on a triple-laser Mo-Flo cell sorter (Cytomation).

### Myocardial Infarction

Mice underwent surgery to ligate the left anterior descending coronary artery (Asahara H et al. J. Rheumatol. 24:430-435 (1997)) as reported previously (Krishnamurthy P et al., Circ. Res. 104:e9-18 (2009)). Cell populations of 2.0×10⁵ mouse EPCs, 2.5×10⁴ or 5×10⁴ CD34+ cells re-suspended in 20 mL PBS were injected intramyocardially into the LV wall (border zone) at two different locations immediately after LAD ligation. Saline group underwent the same surgery but received PBS without cells. Tissue was harvested at days 7, 14 or 28 post-AMI for histological analysis.

### Echocardiography

Transthoracic 2-dimensional M-mode echocardiography was obtained using the Vevo770 (VisualSonics, Toronto, ON, Canada) equipped with a 30-MHz transducer. Mice were anesthetized for analysis with a mixture of 1.5% isoflurane and oxygen (1 L/min) prior to AMI (baseline) and at days 7, 14 and 28 post-AMI M-mode tracings were used to measure LV wall thickness and LV inner diameter in systole and diastole. The mean value of 3 measurements was determined for each sample. Percentage fractional shortening (% FS) and ejection fraction (% EF) were calculated as described previously (Krishnamurthy P et al., FASEB J. 24:2484-2494 (2010)).

### Morphometric Studies

Infarcted hearts were perfused with PBS followed by methanol fixation and paraffin embedding. Morphometric analysis including infarct size and percent fibrotic area was performed on Masson's trichrome-stained tissue sections using ImageJ 1.43u software (US National Institutes of Health).

### Real-Time PCR

RNA was isolated and reverse transcribed into cDNA from sorted EPCs to assess gene mRNA expression of Oct4, Nanog, Sox2, Nk 2.5, connexin43, cardiac troponin T, eNOS and VE cadherin using the Cells to Ct kit (Invitrogen) according to the suggested protocol. Relative mRNA expression of target genes was normalized to the endogenous 18S control gene.

### Chromatin Immunoprecipitation

The ChIP assay was performed as previously described (Liang J et al., Nat. Cell Biol. 10:731-739 (2008); Rajasingh J et al., Circ. Res. 102:e107-117 (2008)).

### Cell Culture and Drug Treatment

Lineage-Sca-1+CD31+ EPCs were cultured on 5 μg/mL human fibronectin coated plates in DMEM supplemented with 10% FBS and penicillin/streptomycin at 37° C. with 5% CO₂. Human CD34+ cells were cultured in X-VIVO (Lonza) media supplemented with 0.5% human serum albumin, 50 ng/mL Flt3 ligand, 20 ng/mL stem cell factor, 50 ng/mL VEGF, and 10 ng/mL thrombopoietin. Either cell type was treated with VPA (2.5 mM) for 24 hours then 500 nM 5′ Aza was added to the culture media and cells remained in culture for an additional 24 hours (48 hours total). Different ranges of doses were tested with above doses being determined as the optimal doses not only for reprogramming but also for displaying minimal cellular toxicity. Further, a staggered treatment protocol, that is, contacting an EPC culture with a first epigenetic programming agent (for example, VPA) for a first period of time (for example, 24 hr), followed by contacting the EPC culture with a second epigenetic programming agent (for example, 5′-Aza) for a second period of time (for example, 24 hr) was determined to be a highly preferred protocol for reprogramming. Mouse EPCs were also cultured with 1 μM BIX-01294 for 48 hours in the same culture conditions.

### Western Blot Analysis

Cell lysate from 1.5×10⁷ SVEC cells was prepared using whole cell lysate buffer (50 mM Tris-HCl, 0.5% Igepal (NP-40), 150 nM NaCl). Proteins (90 μg) were electrophoresed by SDS-PAGE and analyzed using antibodies against acetyl-histone H3K9 (C5B11), di-methyl-histone H3K9 (Cell Signaling), H3 (Cell Signaling), pan acetyl H4 (Active Motif) or H4 (Abcam). Equal protein loading in each lane was verified using antibodies against β-actin and changes in modified histone levels were quantified by first normalization to total H3 or H4 protein.

### Immunofluorescence

Immunofluorescence was performed as previously described (Britten M B et al., Circulation 108:2212-2218 (2003)). Deparaffinized tissue sections were stained for anti-CD31 antibody (BD Biosciences) for capillary density. Donor cells in host tissue were detected by anti-eGFP. Cardiomyocytes were detected by alpha sarcomeric actin antibody (Sigma). Nuclei were counterstained with 4′,6-diamidino-2-phenylindole (DAPI) (1:5000, Sigma Aldrich), and sections were examined with a fluorescent microscope (Nikon ECLIPSE TE200).

### Methylation Analysis by Pyrosequencing

PCR reactions were carried out using the Hotstart Taq polymerase kit (Qiagen). For each PCR reaction, 50 ng of the bisulfite converted DNA was used as a template with 50 μm of forward and reverse primers. After 5 min of initial denaturation at 95° C., the cycling conditions of 44 cycles consisted of denaturation at 95° C. for 15 s, annealing at 65° C. for 30 s and elongation at 72° C. for 45 s. Pyrosequencing was performed using the PyroMark MD Pyrosequencing System (Biotage) as described previously (Lo Celso C et al., J. Vis. Exp. 2007:157 (2007)). Pyrosequencing primer at a concentration of 0.3 μM was annealed to the purified single-stranded PCR product at 28° C. Methylation quantification was performed using the manufacturer-provided software. Methylation studies were performed as previously described (Xie H et al., Nucleic Acids Res. 37:4331-4340 (2009)).

### Microarray Analysis of Human CD34+ RNA

Genome-wide expression analysis was performed on RNA isolated from 1×10⁶ human CD34+ cells cultured 48 hours in X-VIVO (Lonza) media supplemented with 50 ng/mL Flt3L, 20 ng/mL stem cell factor, 50 ng/mL VEGF, 10 ng/mL TPO, 0.5% human serum albumin or 24 hours in the same media+2.5 mM VPA followed by an additional 24 hours with 500 nM 5′-Azacytidine. RNA was isolated and purified using RNA Stat-60 (Tel-test) as per the manufacturer's suggested protocol. RNA quality and purity was assessed using NanoDrop ND-1000. Using the Human HOA5.1 OneAmay (Phalanx Biotech Group), data was collected then analyzed on the Rosetta Resolved System (Rosetta Biosoftware). Differentially expressed gene list was produced by a standard selection criteria as established by Fold Change≧1 and P<0.05. Clustering analysis was performed to visualize the correlations among the replicates and different sample conditions. A subset of 398 genes was selected based on an intensity filter set such that the difference between the maximum and minimum intensity values exceeds 6000 among all microarrays.

### Angiogenesis ELISA Array Analysis of Human CD34+ Cells

Two million human CD34+ cells were cultured as described for 48 hours at 1×10⁶ cells/mL. Conditioned media was first collected by removal of cells by centrifugation (400×g for 15 minutes) then applied to the human angiogenesis array (R&D Systems) as per the manufacturer's suggested protocol. Data were analyzed by ImageJ 1.43u software (NIH) Trends were consistent with a second independent experiment.

### Statistical Analyses

One-tailed, unpaired Student's t tests (Microsoft Excel) were used to measure statistical differences where P<0.05 was considered statistically significant

### Example 2

### Staggered Valproic Acid then 5′-Azacytidine Treatment Results in Genome Wide Enhanced Gene Expression in EPCs

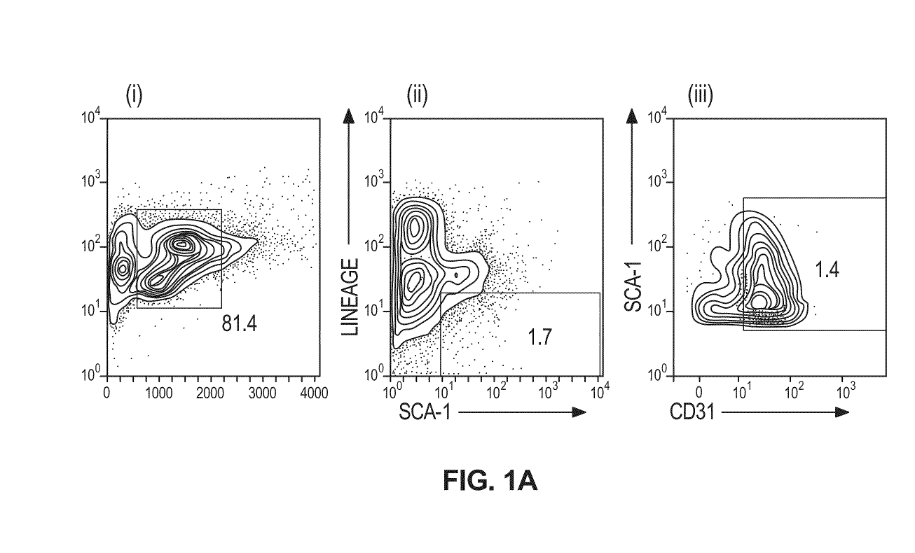

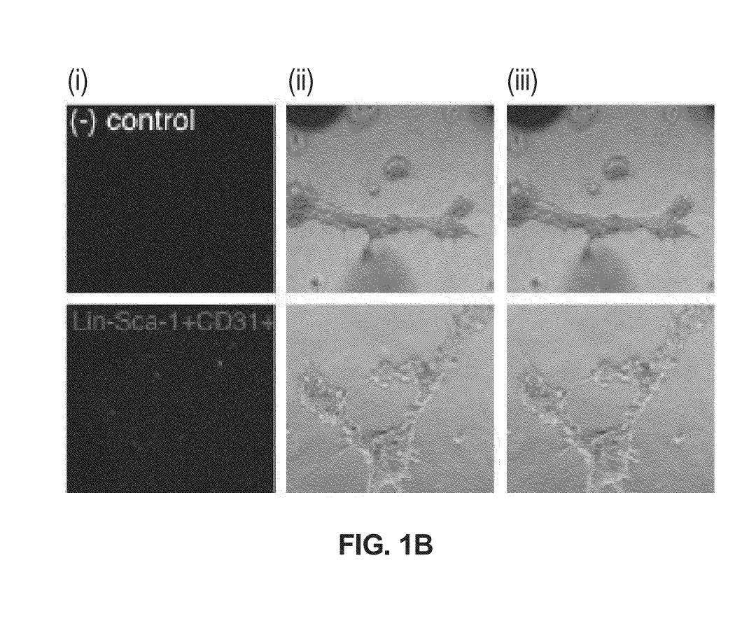

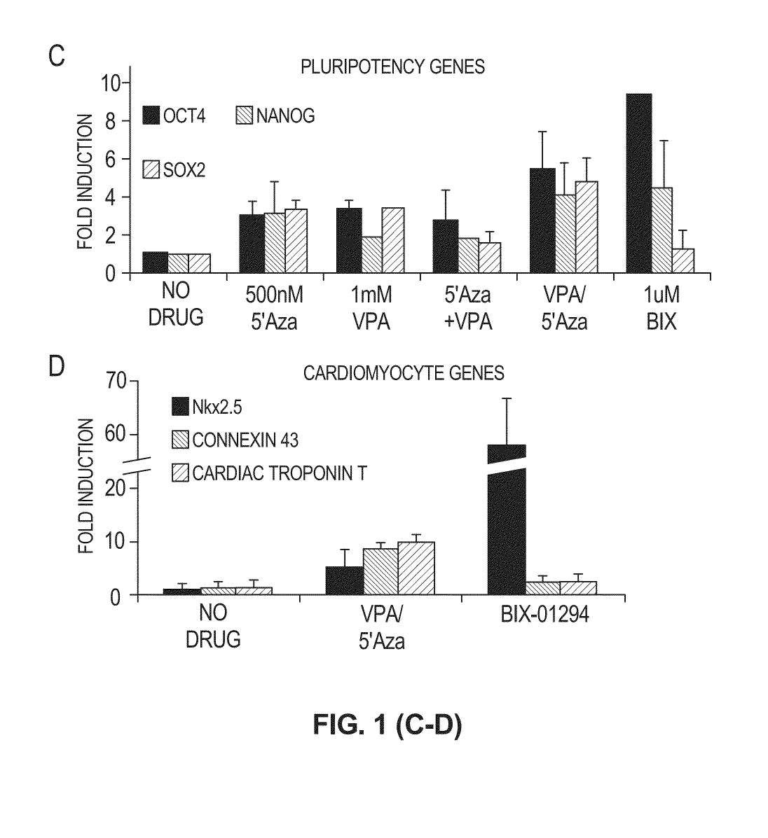

Whole bone marrow was isolated from femurs, tibiae and hip bones of C57BL/6 mice (Lo Celso C et al., J. Vis. Exp. 2007:157 (2007)). Bone marrow mononuclear cells were FACS sorted to greater than 95% purity for the population of cells characterized as Lineage (Lin: CD11b, Ly6G/C, B220, CD3e, Ter119) negative, Sca-1+CD31+, which represents approximately 1.4% of total mononuclear cells (FIG. 1A). This sorting strategy allowed for the isolation of progenitor cell types (Lin-Sca-1+) from the bone marrow with endothelial cell linage (CD31+) (Kim S W et al., J. Am. Coll. Cardiol. 56:593-607 (2010)). Lineage negative Sca-1+CD31+ cells, which will be referred to as EPCs henceforth, showed phenotypic characteristics consistent with their endothelial progenitor identity and incorporated into tubes formed by the mature murine endothelial cell line SVECs on Matrigel (BD Biosciences. FIG. 1B). This suggests that this sorted population encompasses the functional, effector cells found in the bone marrow-derived cultured EPCs without necessitating in vitro culture or differentiation.

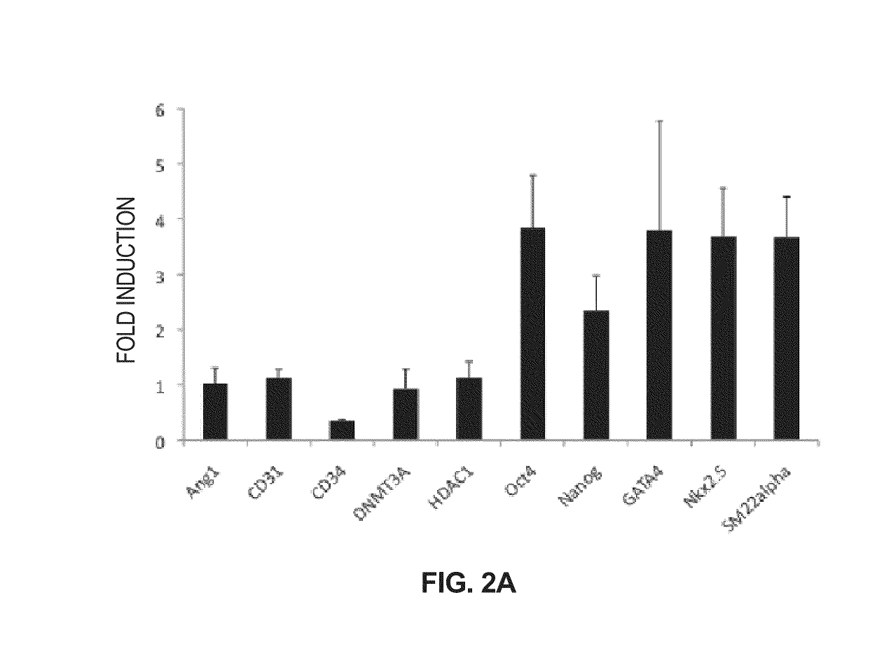

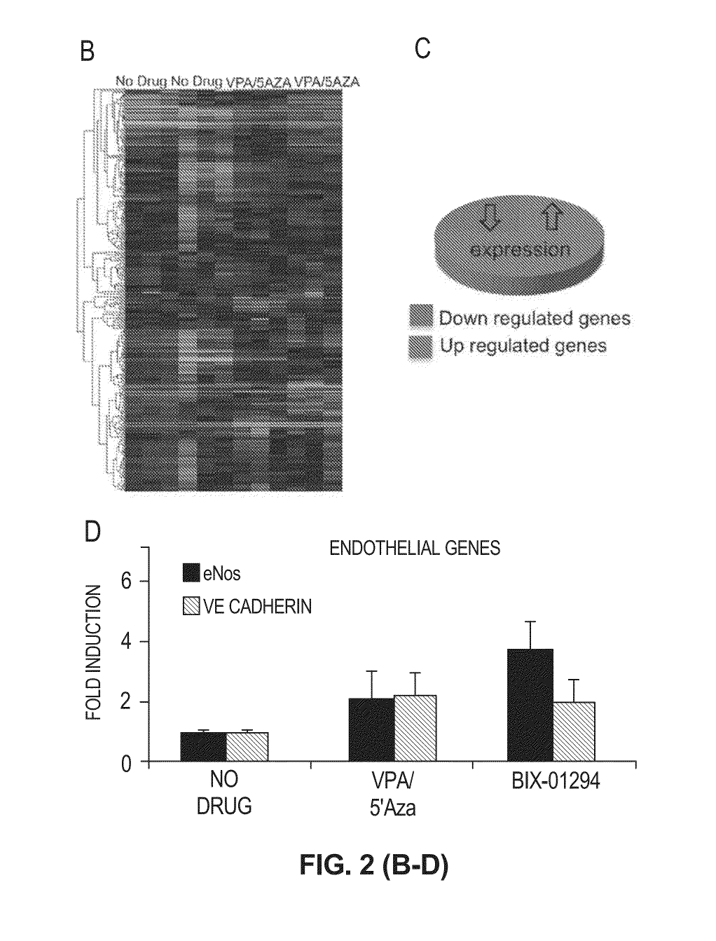

In an attempt to increase their plasticity, 2.0×10⁵ sorted EPCs were seeded on fibronectin coated plates then treated for 48 hours with individual or combinations of epigenetic modifying agents; 500 nM 5′-Azacytidine (5′ Aza; DNA methyltransferase inhibitor), 1 mM valproic acid (VPA; histone deacetylase inhibitor), 1 mM BIX-01294 (histone methyltransferase inhibitor). Drug dosages were verified as non-toxic by cell viability analysis (data not shown). As determined by real-time PCR, this resulted in a significant induction of pluripotency-associated gene expression (Oct4, Nanog and Sox2) with the highest induction in the cells treated for 48 hours with 1 mM BIX-01294 (Oct4 expression: 9.5±2.0 p=0.009), or 24 hours with 1 mM VPA followed by an additional 24 hours with 500 nM 5′ Aza (Oct4 expression: 5.5±1.6 p=0.007) (FIG. 1C). All mRNA expression was normalized to 18S RNA then presented as a fold difference compared to untreated EPCs. Interestingly, one of the most effective conditions included a delayed addition of the DNA methyltransferase inhibitor 5′-Azacytidine, indicating that inhibition of HDAC activity prior to DNA de-methylation may be beneficial for enhanced transcriptional activity throughout the genome. Additionally, real-time PCR results show de novo induction of cardiomyocyte-specific transcripts (FIG. 1D) in the two most effective drug treatment conditions immediately after treatment without necessitating cardiomyocyte differentiation conditions. Since the treated cells express cardiomyocyte genes at basal level they may be more inclined to differentiate into mature CMCs under conditions conducive for CMC differentiation. Similar changes in gene expression were found in human CD34+ cells with the regimen of 2.5 mM VPA followed by 500 nM 5′ Aza (FIG. 2A). BIX-01294 treatment, even at reduced doses, was toxic to human CD34+ cells and was therefore not used in human cell studies.

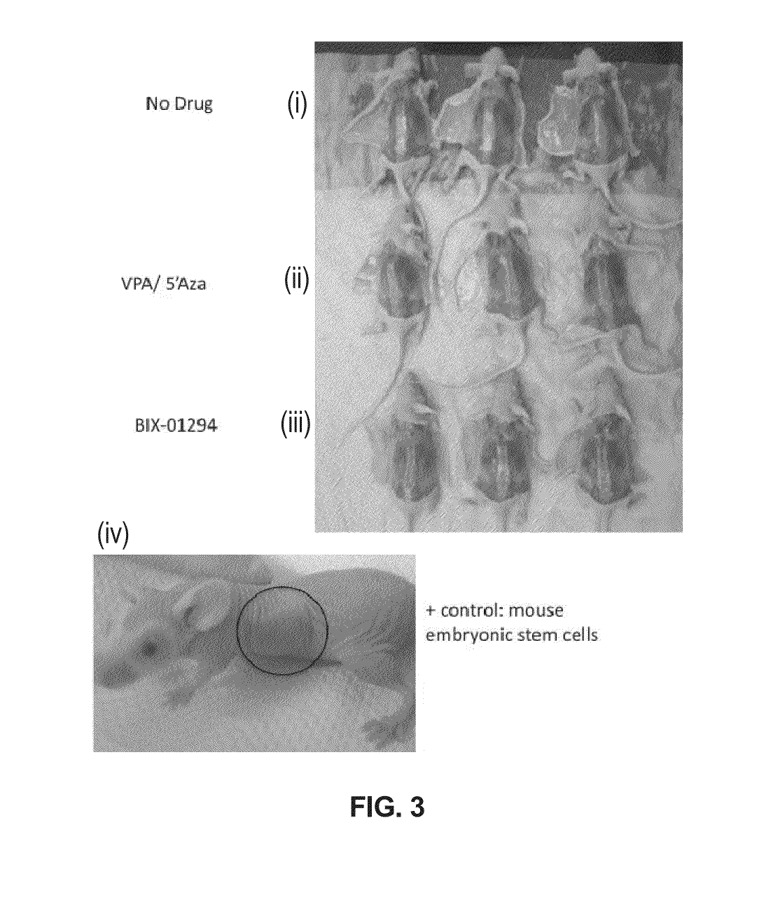

Genome-wide expression profiling of drug-treated human CD34+ cells confirmed that de novo induction of previously silent genes was not limited to any one cell type or cellular function (Suppl Table 1) but was rather reflective of a global transcription permissive chromatin and an open epigenome. Transcriptional profiling of control and treated cells identified 914 genes as significantly up-regulated whereas only 296 genes were significantly down regulated in the staggered VPA/5′ Aza treated cells compared to untreated control CD34+ cells (average of two sample sets run in triplicate, FIGS. 2A, C). These findings indicate that epigenetic modifying drug treatment results in a global increase in gene expression, which is not limited to induction of pluripotency or cardiomyocyte-specific gene expression. Interestingly, endothelial cell specific gene expression (FIGS. 2A, D) in both mouse EPCs and human CD34+ cells treated with drugs was either maintained or was marginally increased suggesting that the cells do not acquire pluripotency and therefore, a potentially tumorigenic phenotype. This was further supported by the inability of the treated cells to form teratomas when injected into immune-deficient mice and followed up to 4 weeks (FIG. 3).

### Example 3

### Chromatin Remodeling Drugs Effectively Remove Targeted Transcription Repressive Epigenetic Marks in Endothelial Cells

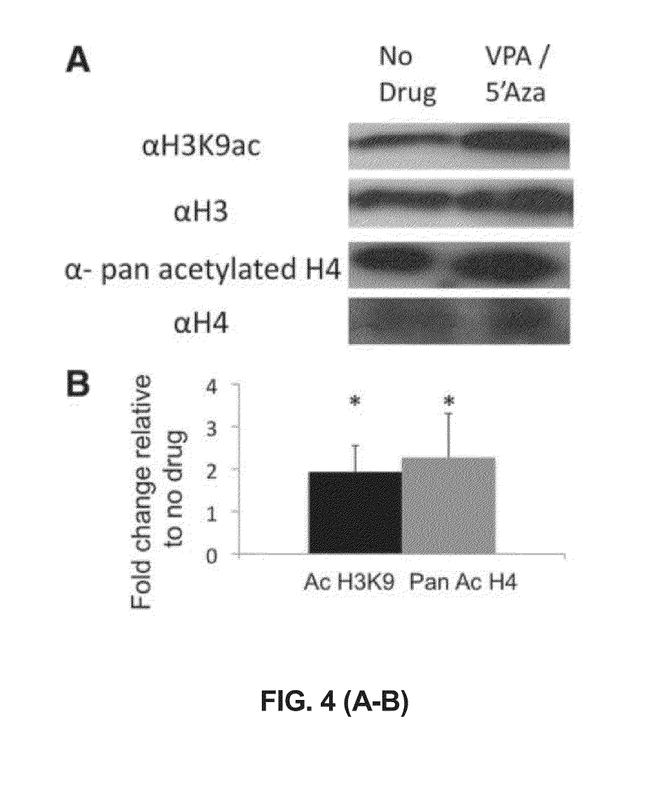

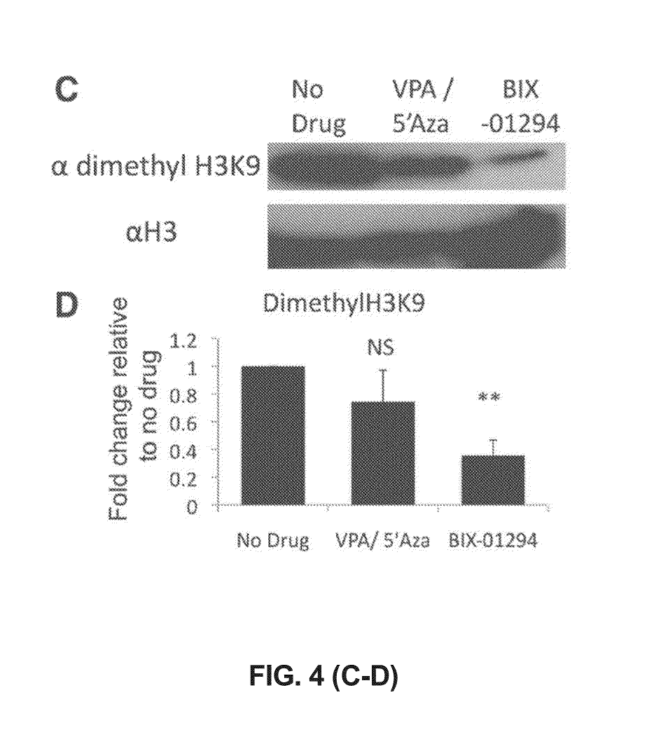

Valproic acid, 5′-Azacytidine and BIX-01294 can be used as effector molecules to remodel the chromatin to allow for less restricted gene expression and increased lineage differentiation potential. VPA and BIX-01294 are direct inhibitors of histone modifying enzymes (histone deacetylase and histone methyltransferase G9a, respectively). Therefore, murine endothelial cells (SVEC) were treated with either 1 μM BIX-01294 or staggered addition of 1 mM VPA then 500 nM 5′-Azacytidine for 48 h and histone acetylation and methylation were assessed by western blot. Acetylation of the lysine 9 position of histone 3 (H3K9, a transcription permissive epigenetic mark) was significantly higher in cells treated with VPA/5′ Aza compared to control cells (FIGS. 4A, B). Additionally, a pan acetylated histone 4 antibody detected a greater than 2-fold increase in total H4 acetylation in the drug treated cells (FIGS. 4A, B). BIX-01294 directly inhibits G9a from forming di-methylated H3K9. H3K9 di-methylation was reduced by 64.3±11% by BIX-01294 compared to untreated control cells (FIGS. 4C, D). This suggests both drug conditions, VPA/5′ Aza and BIX-01294, actively target the intended epigenetic enzymes.

### Example 4

### Drug Treatment Enhances Histone Acetylation at Pluripotency and Cardiac Specific Gene Promoters

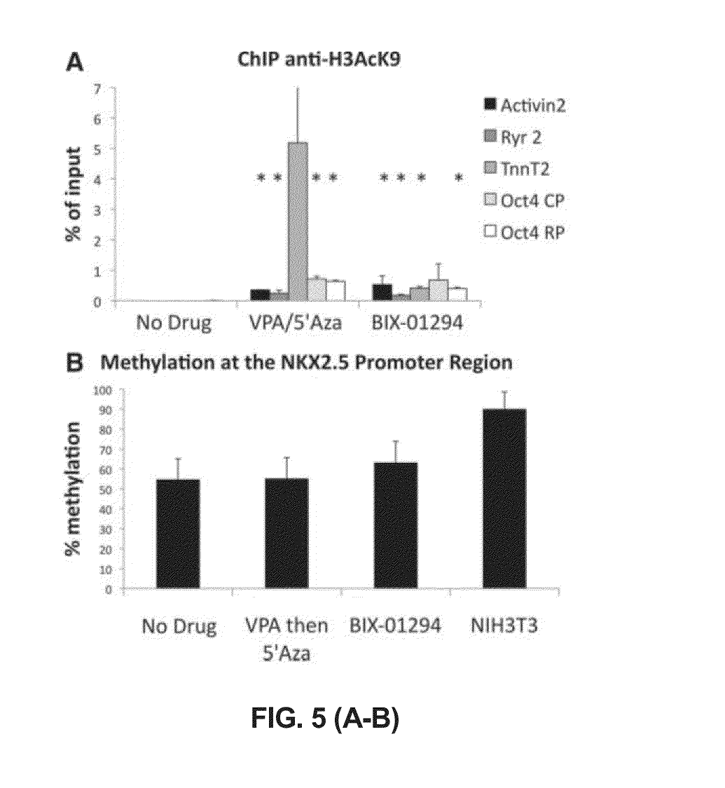

In order to further characterize the epigenetic landscape within the regulatory region of cardiac and pluripotent genes, chromatin immunoprecipitation (ChIP) experiments were carried out to evaluate the level of H3K9-acetylation. Chromatin from control and drug treated SVEC cells was immunoprecipitated with anti-H3K9-acetyl antibodies followed by RT-PCR analysis to quantitate the amount of bound acetylated H3K9 to the Activin 2 (Actn2), Ryanodine receptor (Ryr2), and Troponin T (TnnT2) cardiomyocyte gene promoters or two of the 5′ regulatory regions of Oct4. Cells treated with either BIX-01294 or VPA/5′ Aza showed significant increase in the amount of acetylated H3K9 bound to the core (CP) and regulatory promoter (RP) regions of Oct4 compared to the untreated controls (p<0.05; FIG. 5A). Further, all three cardiac-specific gene promoters analyzed in both drug conditions were more heavily bound by acetylated H3K9 than in the non-treated cells (p<0.05; FIG. 5A). This increase in acetylated H3K9 associated with transcription regulatory regions is potentially responsible for the induction of Oct4 and cardiac-specific gene expression in treated EPCs. DNA methylation analysis of Nkx2.5, a cardiomyocyte-specific transcription factor, showed no significant difference in the CpG methylation patterns between treated and control SVEC cells (FIG. 5B). These data suggest that increased H3K9 acetylation rather than DNA methylation, largely accounts for de novo transcription of these previously silent transcripts.

### Example 5

### Transplantation of Reprogrammed Mouse EPCs Improves Post-Infarct Left Ventricular Function and Adverse Remodeling to a Greater Extent than Untreated EPCs

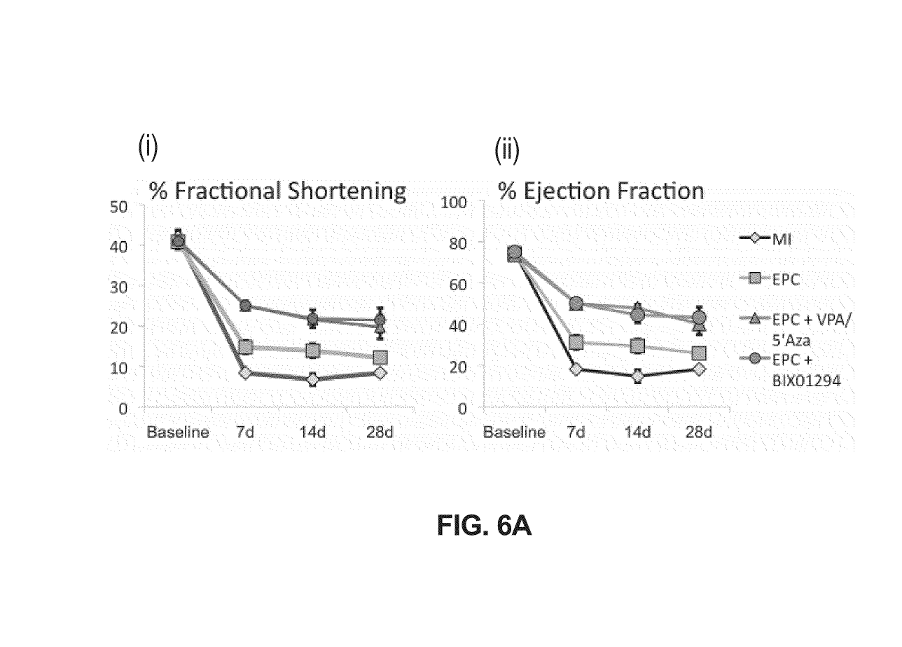

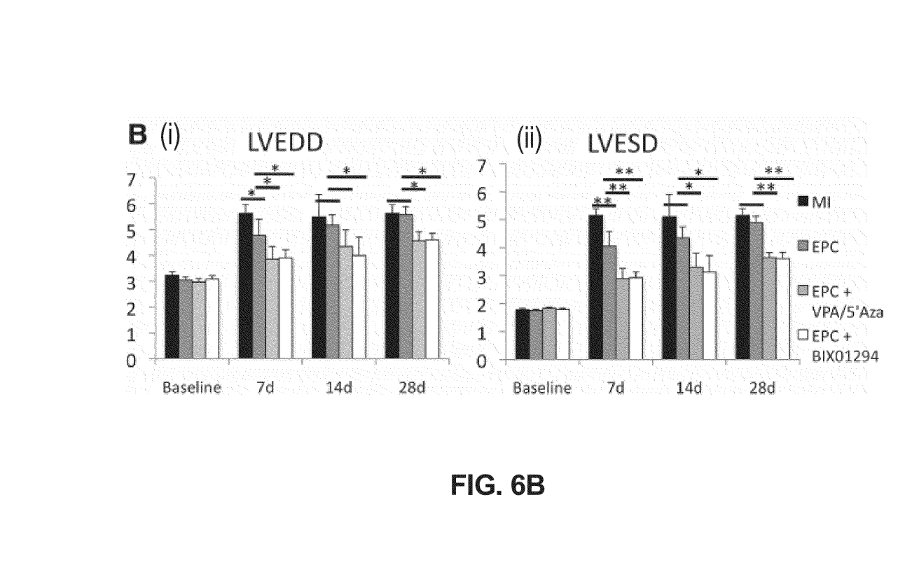

In the next series of experiments, we determined the therapeutic efficacy and differentiation plasticity of drug-treated mouse EPCs in mouse acute myocardial infarction model (AMI). AMI was induced by the permanent ligation of LAD and EPCs, either untreated or treated with BIX-01294 alone or VPA/5′ Aza, were intra-myocardially injected at the ischemic border zone. A subset of mice received saline as control. Left ventricular (LV) functions were evaluated by M-mode echocardiography on days 7, 14 and 28, post-AMI. LV function data indicated a significantly improved ejection fraction (% EF), and fractional shortening (% FS) parameters (p<0.01) in mice receiving drug-treated EPCs compared to untreated EPCs (FIG. 6A). Similarly, left ventricle end-diastolic diameter (LVEDD) and left ventricle end-systolic diameter (LVESD) were significantly reduced in mice receiving treated EPCs compared to control EPCs (p<0.01; FIG. 6B).

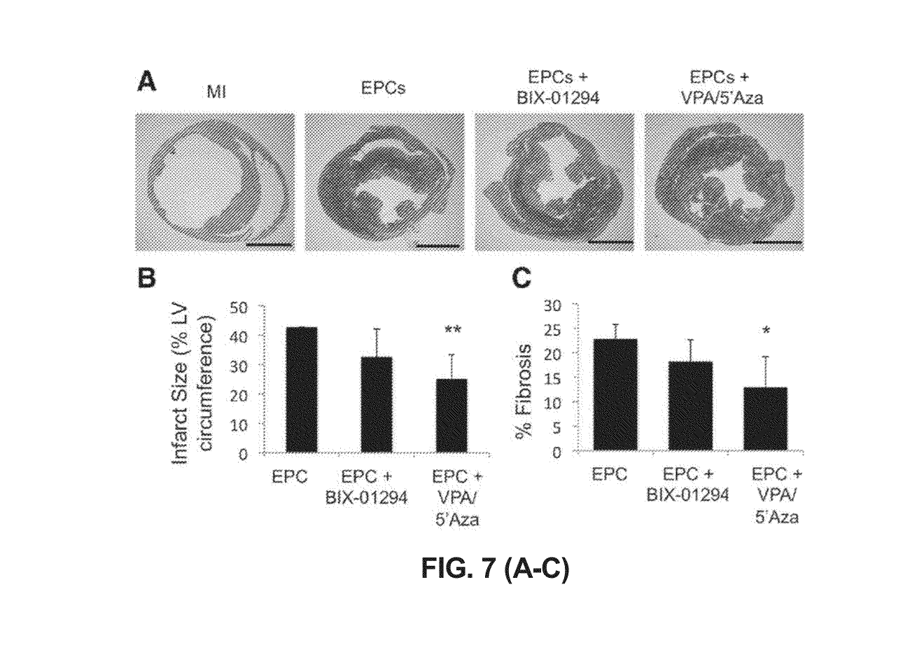

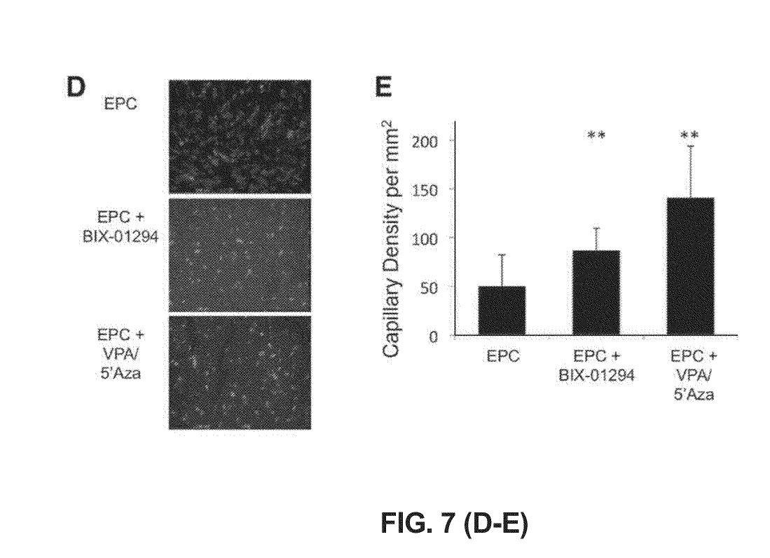

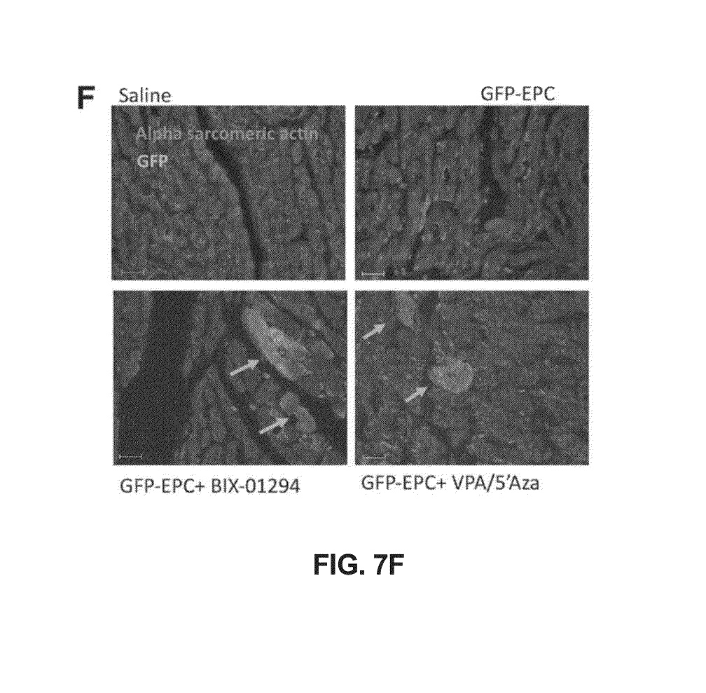

Significant improvement in LV function with drug-treated EPCs was further corroborated by anatomical and histological evidence. An analysis of infarct size and percent LV fibrosis of excised hearts at day 28 post-AMI showed a significant reduction in the infarct size in mice transplanted with drug-treated EPCs, compared to controls (p<0.01; FIGS. 7A, B). Similarly mice receiving drug-treated EPCs showed significantly reduced LV fibrosis on day 28, post-AML (p<0.01; FIG. 7C).

### Example 6

### Epigenetically Reprogrammed EPCs Augment Post-AMI Vascularity and Trans-Differentiate into Cardiomyocytes

Endothelial progenitor cells have been shown to enhance post-infarct LV functions by augmenting neo-vascularization in the ischemic myocardium, largely by paracrine mechanisms. We assessed capillary density in the border zone of ischemic myocardium at 28 days post-AMI and cell injections. Capillaries were identified as vascular structures staining positive for CD31. EPCs reprogrammed with either VPA/5′ Aza or BIX-01294 showed significantly larger number of capillaries, especially in mice receiving EPCs treated with VPA/5′ Aza, when compared to untreated EPCs (FIGS. 7D, E), suggesting that epigenetic reprogramming of EPCs likely enhances their paracrine abilities leading to increased vascularization and concomitant improved LV function.

More interestingly, epigenetic reprogramming of the EPCs with VPA/5′ Aza or BIX-01294 rendered them a more plastic phenotype capable of trans-differentiating to cardiomyocyte lineage, in vivo. eGFP positive Lineage-Sca-1+CD31+ cells were treated with the combination of drugs before transplantation into the ischemic myocardium following AMI. Although GFP+ cells were located in the EPC control group, none were found to co-stain with the cardiomyocyte specific protein marker, alpha-sarcomeric actin. However, some of the BIX-01294-treated EPCs and an even greater number of VPA/5′ Aza treated EPCs co-stained for both GFP and alpha-sarcomeric actin (FIG. 7F). This suggests that, in addition to improving LV function through enhancement of the inherent therapeutic properties of EPCs, treatment of EPCs with VPA/5′ Aza or BIX-01294 also enables acquisition of cardiomyocyte differentiation in vivo.

### Example 7

### Epigenetically Reprogrammed Human CD34+ Cells Display Better Therapeutic Efficacy, Paracrine Activity and Enhance Ischemic Myocardial Vascularity Following AMI in Immune-deficient mice

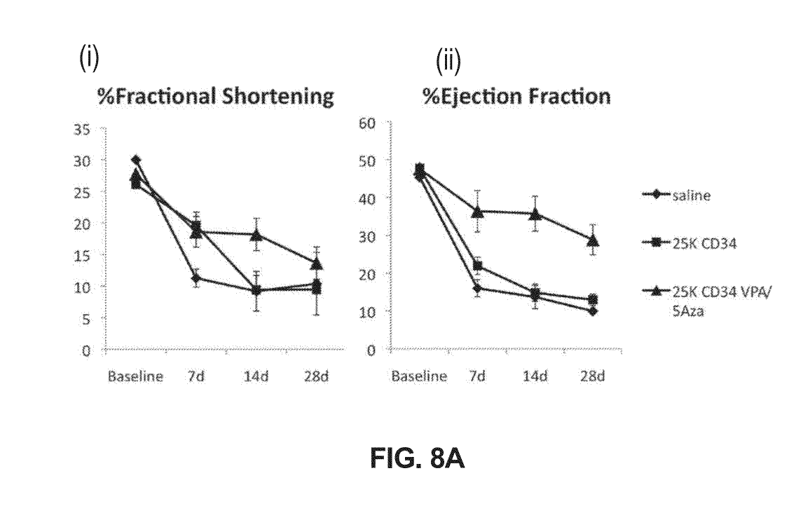

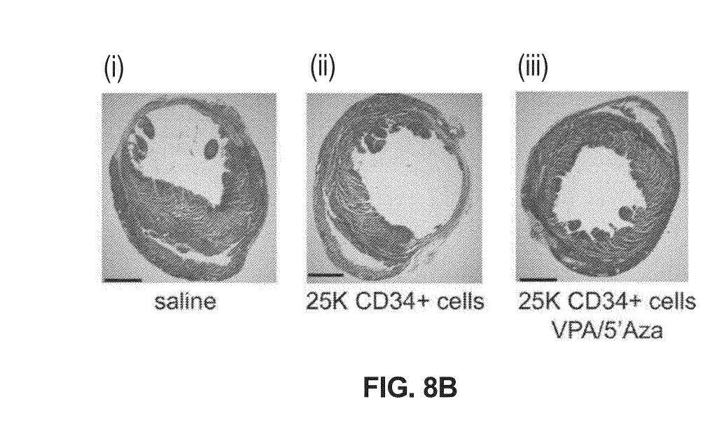

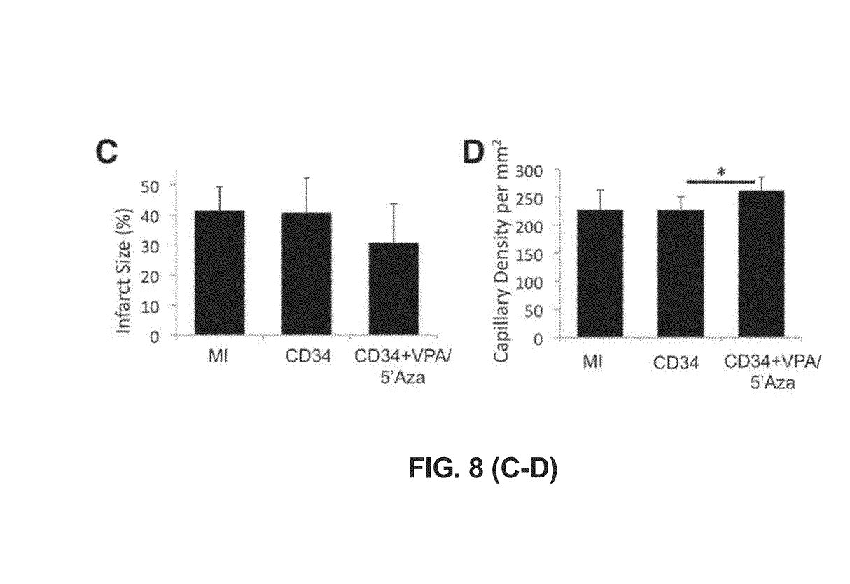

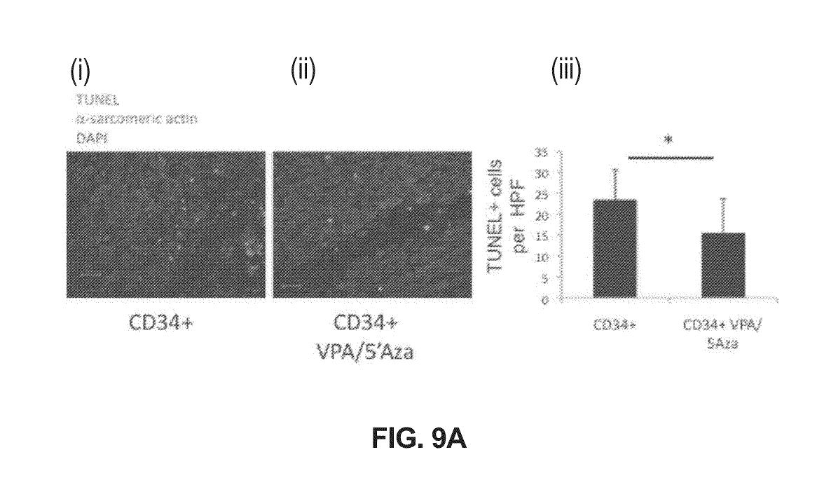

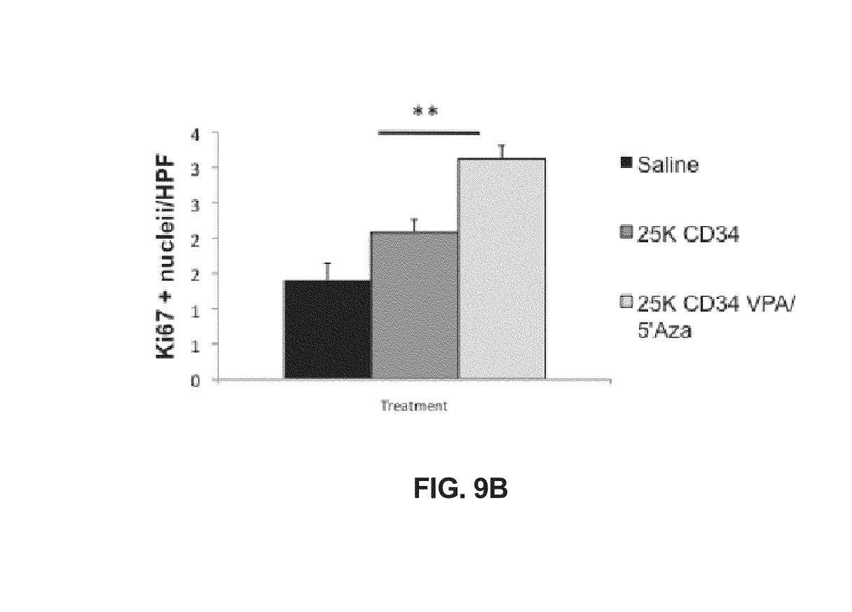

The epigenetic reprogramming by these small molecules was evaluated in human CD34+ cells to determine whether their reprogramming potential is limited to mouse EPCs or whether it can also be translated to human EPCs (bone marrow mobilized CD34+ cells), currently used in clinical trials (Losordo D W et al., Circ. Res. 109:428-436 (2011)). CD34+ cells, obtained from healthy donors and provided by Baxter healthcare, were treated or not with VPA (2.5 mM) and 5′ Aza (500 nM) and mRNA expression of pluripotency and cardiomyocyte-specific transcripts was determined. Despite the promising results seen with BIX-01294 treatment of the mouse EPCs, similar drug doses and even 2-fold reduced levels were toxic to the human CD34+ cells and therefore, omitted. Untreated CD34+ cells did not express significant level of either pluripotent gene or cardiomyocyte gene mRNA, however, VPA/5′ Aza treatment significantly induced the expression of these genes (FIG. 2A). In order to determine enhanced functional capacity of reprogrammed CD34+ cells in the repair of post-infarct myocardium, a previously determined sub-therapeutic dose of 2.5×10⁴ cells was used for post-AMI transplantation in immune-e-deficient mouse AMI model. The dose of 2.5×10⁴ CD34+ cells was chosen deliberately, since our unpublished studies have demonstrated that at this dose, these cells do not confer any therapeutic benefits. The rationale for using a sub-therapeutic dose of cells was to test if the epigenetic reprogramming of cells will enhance the therapeutic efficacy of a cell dose that by itself is not therapeutic. At this dose, mice transplanted with untreated cells did not show any improvement in LV functions, infarct size, or capillary density compared to saline group, whereas transplantation of CD34+ cells treated with VPA/5′ Aza resulted in a significant enhancement of LV function based on % FS and % EF (% EF at d28 CD34+ 12.96±1.49, VPA/5′ Aza 28.82±3.94, p=0.01, FIG. 8A). Additionally, transplantation of reprogrammed CD34+ cells resulted in significantly reduced infarct size and significantly increased capillary density in the border zone of infarcted myocardium (FIG. 8B-D). Additionally, we observed significantly reduced apoptosis and increased proliferation in the infarcted myocardium in the hearts of mice receiving treated CD34+ cells compared to untreated cells (FIGS. 9A, B).

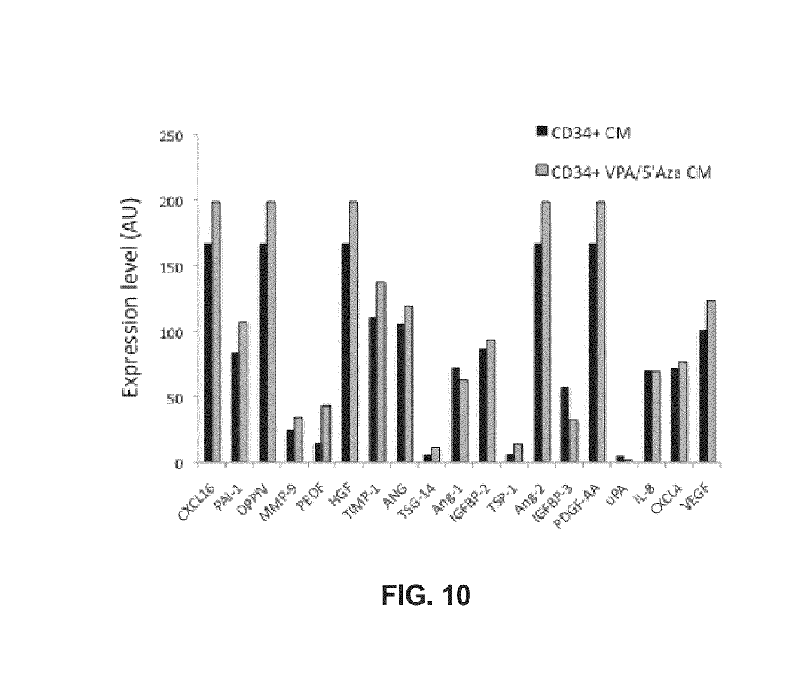

Because the cardio-protective effects (less apoptosis, increased proliferation, increased capillary density, improved overall LV function) in mice that receive VPA/5′ Aza treated CD34+ cells in the AMI model was considerable, we evaluated whether the paracrine secretory profile is enhanced in these treated cells. The Human Angiogenesis Array (R&D Systems) was used to quantitate changes in angiogenesis promoting proteins in the conditioned medium of control and treated cells. CD34+ cells treated with VPA/5′ Aza showed an increase in a majority (15/19) of detected angiogenic proteins (FIG. 10). These data suggests that observed therapeutic effect with treated CD34+ cells in vivo was in part due to their enhanced angiogenic phenotype.

### Example 8

### Treatment with VPA/5′ Aza Confers Cardiac Differentiation Potential to Human CD34+ Cells In Vivo

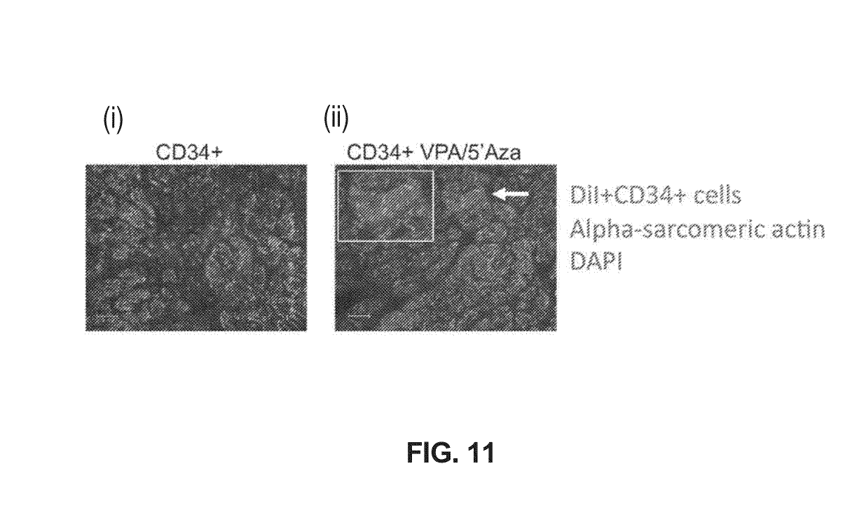

We also determined if increased repair capacity of treated CD34+ cell in vivo also reflect their increased plasticity towards cardiomyocyte differentiation in vivo. AMI was induced in immune-e-deficient mice and 4.0×10⁵ DiI-labeled CD34+ cells with or without treatment with VPA/5′ Aza were injected into the heart at the time of coronary artery ligation. Heart tissue was harvested 7 days post-AMI and stained for alpha-sarcomeric actin while DiI was used as an indicator of donor cells. Immunofluorescence staining identified donor DiI+ cells that co-stained with alpha-sarcomeric actin in hearts exclusively receiving treated CD34+ cells (FIG. 11). In hearts receiving untreated cells, no co-staining was observed. This suggests that in addition to enhancing their angiogenic activity, drug treatment also increased their plasticity towards de-novo cardiomyocyte differentiation.

### Example 9

### Drug Treatment Rescues Angiogenic Dysfunction in Diabetic EPCs

Bone marrow cells were obtained from db/db mice (diabetic) or their heterozygote littermates (healthy). EPCs were isolated by density gradient centrifugation. After washing the cells, they were plated on tissue culture plastic for 30 minutes, the non-adherent cells were then placed on fibronectin-coated plates. Media was changed after 3 days and cells were cultured until day 7. On day 7 cells were washed and treated with 1 mM valproic acid (VPA) for 24 hours at which time cells were washed and then treated with 500 nM 5′ Azacytidine (5′ Aza) for an additional 24 hours. Media from each condition was collected and added with HUVECs to matrigel. HUVECs with media alone served as a control (HUVECS). After 18 hours the total number of tubules formed were counted and normalized to the number of tubules formed in the control condition.

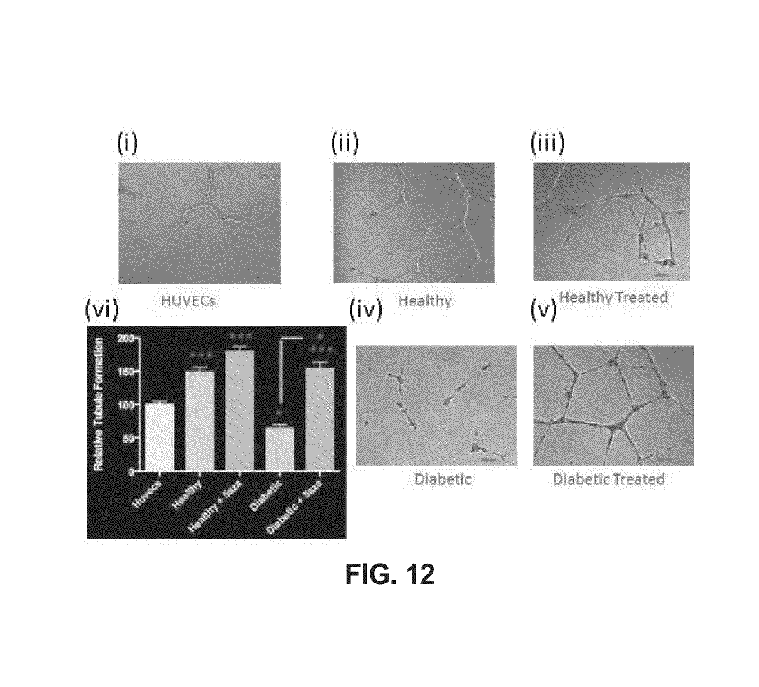

Conditioned media obtained from healthy cells and healthy cells exposed to treatment resulted in a significant increase in tubule formation (that is, angiogenic potential). Conditioned media from diabetic EPCs however, demonstrated a significant decrease in tubule formation when compared to conditioned media from healthy EPCs and healthy treated EPCs. Importantly, conditioned media from diabetic EPCs treated with VPA+5′ Aza significantly enhanced tubule formation to levels obtained from healthy and healthy treated conditioned media (FIG. 12). Taken together, these results suggest that treating diabetic EPCs restores their angiogenic potential.

### Example 10

### Expression of Pro-Angiogenic RNA is Increased in Diabetic EPCs

Db/db diabetic EPCs were obtained and treated as in FIG. 12. After treatment with VPA and 5′ Aza cells were harvested and real time PCR was performed. Results are expressed as a fold increase in treated cells over untreated cells.

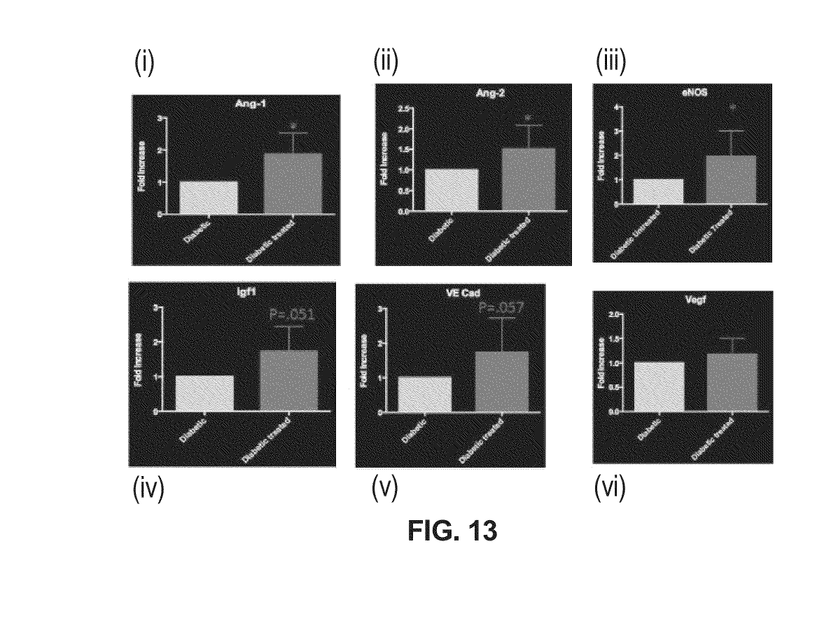

Treatment of EPCs with VPA and 5′ Aza enhanced expression of the pro-angiogenic proteins, Angiopotein 1 (Ang1) which plays a role in blood vessel maturation and stability; Ang2 (angioepotein 2) which is involved in angiogenesis; and endothelial nitric oxide (eNOS) which has a wide range of activity including enhancing angiogenesis in response to ischemia. Further, vascular endothelial cadherin (VE-cad), a marker for endothelial cells, shows a trend towards increased expression (FIG. 13). This suggests the cells are maintaining their endothelial-like phenotype and not reverting back to a more primitive stem cell.

### Example 11

### Treated Diabetic EPCs Restore Blood Flow to Ischemic Hind Limbs

Hind limb ischemia was induced in the left hind limb of diabetic (db/db) mice by resection of the femoral artery. Three days after ligation, laser Doppler was performed to ensure that blood flow was restricted to the limb. After confirmation, the mice were injected in the gastrocnemius muscle with two injections totaling 20 μl of PBS; 1.0×10⁵ diabetic EPCs (in 20 μl of PBS); or 1.0×10⁵ VPA/5′ Aza treated diabetic EPCs (in 201 of PBS). EPCs for injection were obtained and treated as outlined in FIG. 12. Laser Doppler was performed each week for 4 weeks and results are expressed as relative blood flow in the ischemic limb compared to the control limb in each mouse.

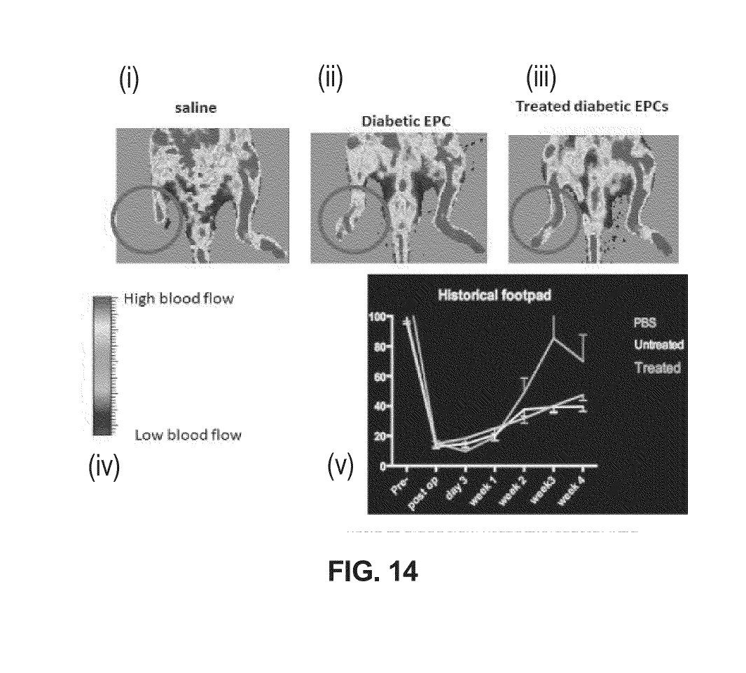

Blood flow was enhanced in mice injected with treated diabetic EPCs when compared to mice that were injected with PBS or untreated diabetic EPCs (FIG. 14). These results suggest that treatment of diabetic EPCs augments their ability to increase angiogenesis.

### Example 12

### Ischemic Limb Capillary Density is Increased in Mice Receiving Treated db/db EPCs

The mice used in FIG. 14 were injected, by tail vein injection, with lectin (a marker for endothelial cells) before sacrifice. Animals were sacrificed on post-operative day 28 and the gastrocnemius muscles were harvested, fixed in methanol, paraffin-embedded, and cross-sectioned. Nuclei are stained in blue (dapi) and endothelial cells in red (lectin).

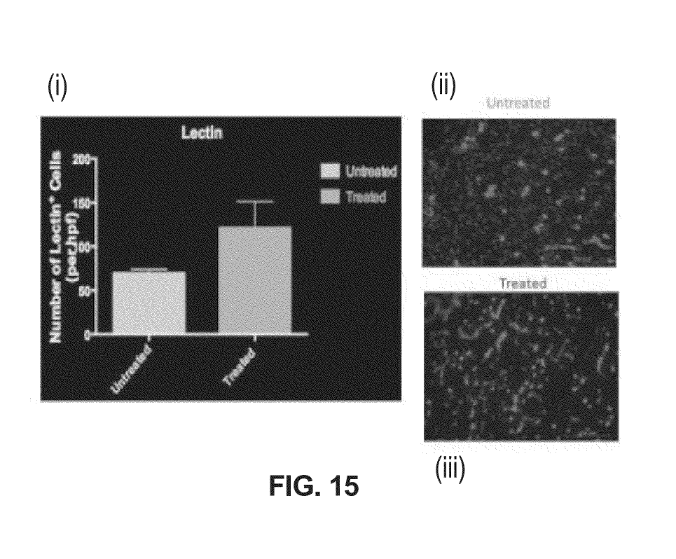

An increase in lectin positive cells was observed in the gastrocnemius muscle of mice injected with EPCs treated with VPA and 5′ Aza when compared to those injected with untreated EPCs (FIG. 15). Quantification is shown on the left (subpanel (i)) while sample sections are on the right (subpanels (ii) and (iii)). These results suggest that injection with treated EPCs enhances blood vessel formation in response to ischemia in diabetic mice.

### Example 13

### Injection of Treated Diabetic EPCs Preserves Muscle Architecture in Ischemic Limbs

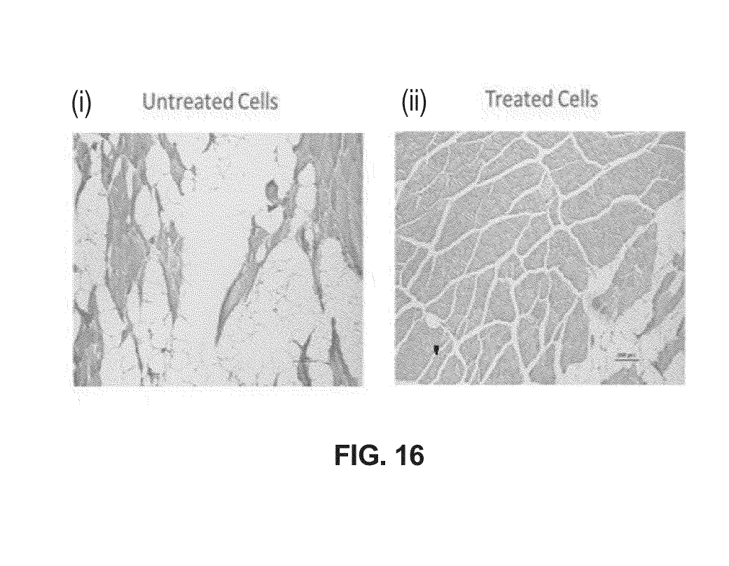

Gastrocnemius muscle samples were obtained as in FIG. 15 and stained with hemotoxylin and eosin. Increased fat infiltration was observed in the muscles of muscles injected with untreated EPCs while mice injected with VPA+5′ Aza-treated EPCs exhibit less fat infiltration (FIG. 16). These results suggest injection with treated EPCs preserves muscle structure in response to ischemic injury when compared to treatment with untreated diabetic EPCs.

### Example 14

### 5-Aza/VPA Treatment Increases the Secretion of Angiogenic Cytokines by Human CD34+ Cells

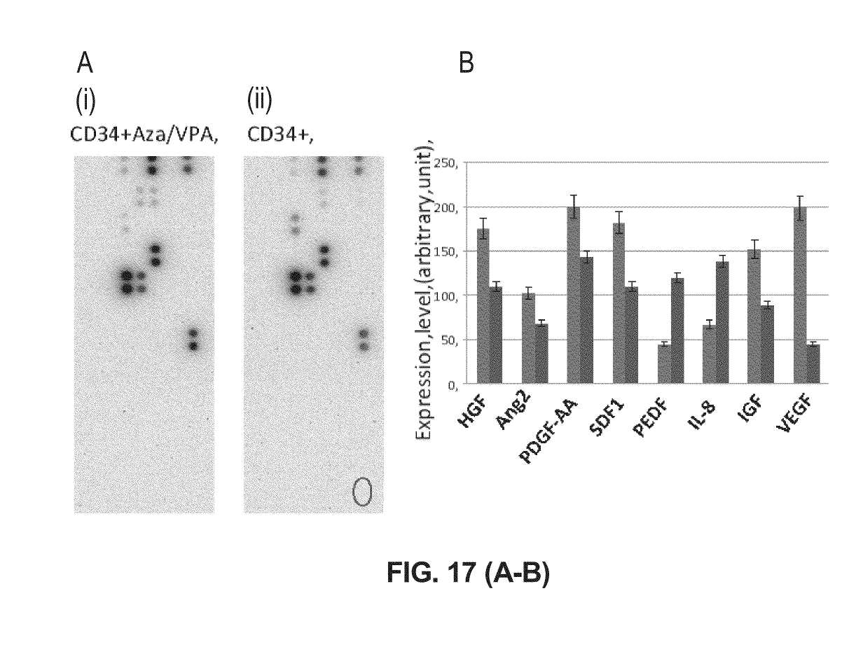

Cultured CD34+ cells were treated or not with 2.5 mM (the best dose for cell response determined by culturing the cell with doses ranging from 500 nM-5 mM) VPA for 24 h followed by additional 24 h with 500 nM 5′ Aza. As shown in FIG. 17A, conditioned medium from cells treated (subpanel (i)) or untreated (subpanel (ii)) was assayed on protein arrays. Drug treatment enhanced the expression of most angiogenic cytokines (blue bars) (FIG. 17B).

### REFERENCES

All patents, patent applications, patent application publications and other publications that are cited herein are hereby incorporated by reference as if set forth in their entirety.

It should be understood that the methods, procedures, operations, composition, and systems illustrated in figures. may be modified without departing from the spirit of the present disclosure. For example, these methods, procedures, operations, devices and systems may comprise more or fewer steps or components than appear herein, and these steps or components may be combined with one another, in part or in whole.

Furthermore, the present disclosure is not to be limited in terms of the particular embodiments described in this application, which are intended as illustrations of various embodiments. Many modifications and variations can be made without departing from its scope and spirit. Functionally equivalent methods and apparatuses within the scope of the disclosure, in addition to those enumerated herein, will be apparent to those skilled in the art from the foregoing descriptions.

### TERMINOLOGY AND DEFINITIONS

The terminology used herein is for the purpose of describing particular embodiments only, and is not intended to be limiting. With respect to the use of substantially, any plural and/or singular terms herein, those having skill in the art can translate from the plural as is appropriate to the context and/or application. The various singular/plural permutations may be expressly set forth herein for the sake of clarity.

Terms used herein are intended as “open” terms (e.g., the term “including” should be interpreted as “including but not limited to,” the term “having” should be interpreted as “having at least,” the term “includes” should be interpreted as “includes but is not limited to,” etc.).

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms, For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into subranges as discussed above.

A range includes each individual member. Thus, for example, a group having 1-3 members refers to groups having 1, 2, or 3 members. Furthermore, a range includes any conceivable value or fraction contained therein, whether explicitly stated in the specified range in the disclosure. Thus, for example, range of a time period from about 1 hr to about 96 hrs includes any whole integer or fractional value of an integer between 1 hr and 72 hrs, including 1 hr and 72 hr, such as 2.8 hr, 40 hr, 56.5 hr, and so forth. The same meaning of range is applicable to a range of concentrations and a range of amounts. For example, an epigenetic modifier in the range from about 100 nM to about 5 mM includes about 100 nM and about 5 mM, as well as about 205 nM, about 1.25 mM, about 23 mM and so forth. For example, an amount of cells in the range from 10⁴ cells to 10⁶ cells includes 10 cells, 10⁶ cells, as well as 2.75×10⁴ cells, 4.26×10⁵ cells, 9.03×10⁵ cells and so forth. Similarly, a group having 6 members refers to groups having 1, 2, 3, 4, or 6 members, and so forth.

The use of treat, treated, treating and treatment when referring to manipulation of cells in vivo or ex vivo with a reprogramming agent, such as an epigenetic modifier, means that cells are contacted with a reprogramming agent. The forms of cell contact with a reprogramming agent include exposing cells to a culture medium or incubating cells with a culture medium, wherein the culture medium includes a reprogramming agent.

As used herein, “diseased cell,” “diabetic cell,” and “aged cell” refer to cells obtained from bone marrow of a subject having a disease, a subject having a form of diabetes (for example, Type I Diabetes or Type II diabetes), or a subject who is elderly, respectively.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

The invention claimed is:

1. A reprogrammed endothelial progenitor cell, said cell comprises a bone marrow-derived cell expressing the CD34+ marker and at least one cardiomyocyte-specific gene.

2. The reprogrammed endothelial progenitor cell of claim 1, wherein said cell lacks teratoma potential.

3. A method of reprogramming an endothelial progenitor cell, said method comprises contacting a bone marrow-derived, CD34+ cell with a first reprogramming agent.

4. The method of claim 3, wherein the first reprogramming agent comprises an epigenetic modifier.

5. The method of claim 4, wherein the epigenetic modifier is selected from the group consisting of Trichostatin A, valproic acid, 5′-Azacytidine and BIX-01294.

6. The method of claim 3, further comprising a second reprogramming agent.

7. The method of claim 6, further comprising forming an admixture of the first and second reprogramming agents prior to contacting the bone marrow-derived, CD34+ cell.

8. The method of claim 6, wherein the bone marrow-derived, CD34+ cell is contacted with the first programming agent for a first time period, followed by the bone marrow-derived, CD34+ cell being contacted with the second reprogramming agent for a second time period.

9. The method of claim 8, wherein the first reprogramming agent comprises valproic acid and the second reprogramming agent comprises 5′-Azacytidine.

10. The method of claim 9, wherein the bone marrow-derived, CD34+ cell is contacted with from about 1.0 mM to about 5.0 mM valproic acid ranging from about 1 hr to about 48 hr followed by the addition of from about 250 nM to about 1.0 mM 5′-Azacytidine ranging from about 1 hr to about 48 hr.

11. The method of claim 9, wherein the bone marrow-derived, CD34+ cell is contacted with about 2.5 mM valproic acid for about 24 hr followed by the addition of about 500 nM to 5′-Azacytidine ranging for about 24 hr.

12. The method of claim 3, wherein the bone marrow-derived, CD34+ cell is obtained from a subject selected from the group consisting of a healthy subject, an elderly subject and a subject having a disease.

13. The method of claim 3, wherein the bone marrow-derived, CD34+ cell is obtained from a subject selected from an elderly subject.

14. The method of claim 3, wherein the bone marrow-derived, CD34+ cell is obtained from a subject having a disease.

15. The method of claim 14, wherein the disease comprises a form of diabetes.

16. The method of claim 3, further comprising the steps of isolating the bone marrow-derived, CD34+ cell from bone marrow-derived population of cells with a cell sorting technique.

17. The method of claim 16, wherein the cell sorting technique is fluorescent activated cell sorting.

18. A method of improving an ischemic condition, said method comprises contacting the ischemic condition with a therapeutically effective amount of reprogrammed endothelial progenitor cells, said cells comprises bone marrow-derived cells expressing the CD34+ marker and at least one cardiomyocyte-specific gene.

19. The method of claim 18, wherein the ischemic condition is selected from an ischemic-damaged non-cardiotissue or an ischemic-damaged myocardium.

20. The method of claim 18, wherein the ischemic condition comprises an ischemic-damaged myocardium.

21. The method of claim 18, wherein the therapeutically effective amount of reprogrammed endothelial progenitor cells comprises an amount from about 1×10⁴ cells to about 1×10⁶ cells per kg body weight.

22. The method of claim 18, wherein the therapeutically effective amount of reprogrammed endothelial progenitor cells are administered by injection.

23. A pharmaceutical composition comprising
a population of reprogrammed endothelial progenitor cells comprising bone marrow-derived cells expressing the CD34+ marker and at least one cardiomyocyte-specific gene; and
a physiologically acceptable buffer.

24. A kit for preparing a reprogrammed endothelial progenitor cell, wherein said cell comprising a bone marrow-derived cell expressing the CD34+ marker and at least one cardiomyocyte-specific gene, said kit comprises reprogramming agents and optionally instructions.
